(19) United States
(12) Patent Application Publication
(10) Pub. No.: US 2008/0081815 A1
(43) Pub. Date: Apr. 3, 2008

(54) Heteroaryl-pyrazole derivatives as cannabinoid CB1 receptor antagonists

(75) Inventors: Jinhwa Lee, Yongin-si (KR); Jeong Min Kim, Seoul (KR); Chong-Hwan Jonathan Chang, Yongin-si (KR); Suk Youn Kang, Yongin-si (KR); Hee Jeong Seo, Seoul (KR); Kwang-Seop Song, Yongin-si (KR); Sung-Han Lee, Seoul (KR); Kwang-Woo Ahn, Seoul (KR)

Correspondence Address: ANDERSON, KILL & OLICK, P.C., 1251 AVENUE OF THE AMERICAS, NEW YORK,, NY, 10020-1182, US

(73) Assignee: GREEN CROSS Corporation, Yongin-si (KR)

(21) Appl. No.: 11/541,269

(22) Filed: Sep. 29, 2006

(51) Int. Cl. A61K 31/501; A61K 31/497; A61K 31/433; A61K 31/4245; A61K 31/4196; C07D 413/14; C07D 417/14; C07D 403/14

(52) U.S. Cl. 514/252.05; 544/238; 544/405; 546/275.7; 548/137; 548/143; 514/255.05; 514/341; 514/362; 514/364; 514/381; 514/383; 548/252; 548/266.2

(57) ABSTRACT

A novel heteroaryl-pyrazole compound of formula (I) or a pharmaceutically acceptable salt thereof is effective as a cannabinoid CB₁ receptor inverse agonist or antagonist, which is useful for preventing or treating obesity and obesity-related metabolic disorders. The prevention also provide a method for preparing same, a pharmaceutical composition containing same, and a method for preventing or treating obesity and obesity-related metabolic disorders.

### FIELD OF THE INVENTION

The present invention relates to a novel heteroaryl-pyrazole compound which is effective as a cannabinoid CB₁ receptor inverse agonist or antagonist.

### DESCRIPTION OF THE PRIOR ART

The World Health Organization (WHO) recently reported that obesity has become a global epidemic, posing a serious threat to public health because of the increased risk of associated health problems (See Report of a WHO Consultation on Obesity: Obesity-Preventing and Managing a Global Epidemic; World Health Organization: Geneva, 1997). Obesity is characterized by excess body fat, especially visceral fat, and constitutes a pro-inflammatory state eventually leading to serious health consequences. There are growing evidences that obesity as a chronic disease cannot be cured by short-term dieting or exercise alone, but additional pharmacological treatments would lead to higher success rates.

CB₁ cannabinoid receptor belongs to G-protein-coupled receptor (GPCR) type and is coupled to inhibitory G proteins (G(i/o)) to inhibit certain adenylyl cyclase isozymes, leading to decreased cAMP production, decreased Ca²⁺ conductance, increased K⁺ conductance, and increased mitogen-activated protein kinase activity (See Di Marzo et al., Nat. Rev. Drug Discovery 2004, 3, 771-784; Rhee, M. H. et al., J. Neurochem. 1998, 71, 1525-1534). The major physiological effect of cannabinoids (in the central nervous system (CNS) and neuronal tissues) is the modulation of neurotransmitter release via activation of presynaptic CB₁ receptors located on distinct types of axon terminals throughout the brain (See Howlett, A. C. et al., Neuropharmacology 2004, 47 (Suppl. 1), 345-358).

The CB₁ receptor is mainly expressed in several brain areas including the limbic system (amygdala, hippocampus), hypothalamus, cerebral cortex, cerebellum, and basal ganglia. In the cerebellum and basal ganglia cannabinoids modulate the locomotor activity. In the limbic system, cannabinoids influence learning, memory, emotion, and motivation, and through activation of CB₁ receptors in the limbic system-hypothalamus axis, cannabinoids have an important role in the control of appetite. Moreover, lower levels of CB₁ receptors can also be found in peripheral tissues including urinary bladder, testis, prostate, GI tract, heart, lung, adrenal gland, parotid gland, bone marrow, uterus, ovary, and adipose tissue (See Cota, D. et al., J. Clin. Invest. 2003, 112, 423-431; Ravinet Trillou, C. et al., Int. J. Obes. Relat. Metab. Disord. 2004, 28, 640-648; Galiegue, S. et al., Eur. J. Biochem. 1995, 232, 54-61; Howlett, A. C. et al., Pharmacol. Rev. 2002, 54, 161-202).

Many preclinical in vitro and in vivo experiments have been shown that CB₁ receptor antagonists can influence energy homeostasis by central and peripheral mechanisms and may represent promising targets to treat diseases that are characterized by impaired energy balance. Already the first published studies with rimonabant (SR141716) in both rodents (See Arnone, M. et al., Psychopharmacology (Berlin) 1997, 132, 104-106) and primates (See Simiand, J Keane, M.; Keane, P. E.; Soubrie, P. Behav. Pharmacol. 1998, 9, 179-181) showed clear differentiation, i.e., marked effects on sweet food intake versus marginal effects on regular chow intake or water drinking. Many other preclinical “proof of concept” studies have been performed in the meantime with several CB agonists and antagonists to further uncover the amount and mode of contribution of cannabinergic system modulators to energy homeostasis. Almost all of those studies have been recently reviewed (ee Smith, R. A. et al., IDrugs 2005, 8, 53-66).

Considering the important impact of obesity on public health and the lack of any efficient and viable drug to cure it, it is no surprise that CB₁ antagonists are currently the subject of intense studies, which were published in several reviews (See Adam, J. et al., Expert Opin. Ther. Patents, 2002, 12(10), 1475-1489; Hertzog, D. L. Expert Opin. Ther. Patents, 2004, 14(10), 1435-1452; Lange, J. H. M. et al., Drug Discov. Today, 2005, 10, 693-702; Bishop, M. J. J. Med. Chem., 2006, 49(14), 4008-4016).

### SUMMARY OF THE INVENTION

It is a primary object of the present invention to provide a novel heteroaryl-pyrazole compound of formula (I) or a pharmaceutically acceptable salt thereof, which is effective as a cannabinoid CB₁ receptor inverse agonist or antagonist, useful for preventing or treating obesity and obesity-related metabolic disorders.

It is another object of the present invention to provide a method for preparing the inventive compound.

It is another object of the present invention to provide a pharmaceutical composition for preventing or treating obesity and obesity-related metabolic disorders, comprising the inventive compound as an active ingredient.

### DETAILED DESCRIPTION OF THE INVENTION

In accordance with one aspect of the present invention, there is provided a compound of formula (I) or a pharmaceutically acceptable salt thereof and a method for preparing same:

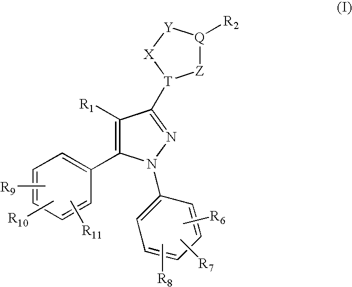

wherein:

R₁ is hydrogen, C₁₋₅ alkyl, substituted C₁₋₅ alkyl, C₂₋₄ alkenyl, substituted C₂₋₄ alkenyl, C₂₋₄ alkynyl, substituted C₂₋₄ alkynyl, or (CH₂)ₙ—C₃₋₅ carbocycle, n being 0 or 1;

R₂ is hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2;

R₃ and R₄ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or

R₃ and R₄, together with the nitrogen atom to which they are bonded, form a 4- to 10-membered saturated or unsaturated heterocyclic ring which is optionally substituted by one or more C₁₋₃ alkyl, benzyl, phenyl, C₁₋₃ alkoxy or halogen;

R₅ is phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutes;

R₆, R₇, R₈, R₉, R₁₀ and R₁₁ are each independently hydrogen, halogen, C₁₋₃ alkyl, C₁₋₃ alkoxy or trifluoromethyl;

X, Y and Z are each independently selected from the group consisting of —C(R₁₂)═, —O—, —N═, —N(R₁₃)— and —S— to form an aromatic heterocycle together with Q and T;

Q and T are each independently

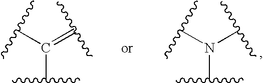

with the proviso that both Q and T can not be simultaneously

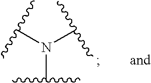

R₁₂ and R₁₃ are each independently hydrogen, carbocycle, substituted carbcycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, C₂₋₆ alkynyl optionally substituted by alkoxy or halogen, (CH₂)ₘ—C₃₋₆ carbocycle optionally substituted by alkoxy or halogen, or (CH₂)ₘ—R₅, m being 1 or 2, and R₅ having the same meaning as defined above.

The aromatic heterocycles formed by X, Y, Z, Q and T encompass, for example, oxazole, isoxazole, thiazole, isothiazole, triazole, oxadiazole, thiadiazole and tetrazole.

As used herein, the term “alkyl” refers to a straight or branched chain saturated hydrocarbon radical. Examples of “alkyl” as used herein include, but are not limited to, methyl, ethyl, n-propyl, isopropyl, n-butyl, isobutyl, t-butyl, n-pentyl, isopentyl and hexyl.

As used herein, the term “substituted alkyl” refers to a straight or branched chain saturated hydrocarbon radical, which is optionally substituted by one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heteroaryloxy, heterocyclic, aminosulfonyl, sulfonylamino, carboxyamide, ureido, nitro, cyano and halogen.

As used herein, the term “alkenyl” refers to a straight or branched chain hydrocarbon radical having at least one carbon-carbon double bond. Examples of “alkenyl” as used herein include, but are not limited to, ethenyl and propenyl.

As used herein, the term “substituted alkenyl” refers to a straight or branched chain hydrocarbon radical having at least one carbon-carbon double bond, which has optional substituents selected from the group consisting of C₁₋₃ alkyl optionally having one to three fluorine substituents, amino, aryl, cyano and halogen.

As used herein, the term “alkynyl” refers to a straight or branched chain hydrocarbon radical having at least one carbon-carbon triple bond. Examples of “alkynyl” as used herein include, but are not limited to, acetylenyl and 1-propynyl.

As used herein, the term “substituted alkynyl” refers to a straight or branched chain hydrocarbon radical having at least one carbon-carbon triple bond, optionally having one or more substituents selected from the group consisting of C₁₋₃ alkyl optionally having one to three fluorine substituents, amino, aryl and halogen.

As used herein, the term “halogen” refers to fluorine (F), chlorine (Cl), bromine (Br), or iodine (I).

As used herein, the term “carbocycle” refers to a non-aromatic cyclic hydrocarbon radical composed of three to seven carbon atoms. Five- to seven-membered rings may contain a double bond in the ring structure. Exemplary “carbocycle” groups include, but are not limited to, cyclopropyl, cyclobutyl, cyclopentyl, cyclopentenyl, cyclohexyl, and cycloheptyl.

As used herein, the term “substituted carbocycle” refers to a non-aromatic cyclic hydrocarbon radical composed by three to seven carbon atoms, which is optionally substituted with one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heterocyclic, aminosulfonyl, sulfonylamino, carboxyamide, nitro, ureido, cyano and halogen.

As used herein, the term “aryl” refers to an optionally substituted benzene ring or refers to a ring system which may result by fusing one or more optional substituents. Exemplary optional substituents include substituted C₁₋₃ alkyl, substituted C₂₋₃ alkenyl, substituted C₂₋₃ alkynyl, heteroaryl, heterocyclic, aryl, alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, or ureido. Such a ring or ring system may be optionally fused to aryl rings (including benzene rings) optionally having one or more substituents, carbocycle rings or heterocyclic rings. Examples of “aryl” groups include, but are not limited to, phenyl, naphthyl, tetrahydronaphthyl, biphenyl, indanyl, anthracyl or phenanthryl, as well as substituted derivatives thereof.

As used herein, the term “heteroaryl” refers to an optionally substituted monocyclic five to six-membered aromatic ring containing one or more heteroatomic substitutions selected from S, SO, SO₂, O, N, or N-oxide, or refers to such an aromatic ring fused to one or more rings such as heteroaryl rings, aryl rings, heterocyclic rings, or carbocycle rings (e.g., a bicyclic or tricyclic ring system), each having optional subsituents.

Examples of optional substituents are selected from the group consisting of substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen or ureido. Examples of “heteroaryl” groups used herein include, but are not limited to, benzoimidazolyl, benzothiazolyl, benzoisothiazolyl, benzothiophenyl, benzopyrazinyl, benzotriazolyl, benzo[1,4]dioxanyl, benzofuranyl, 9H-a-carbolinyl, cinnolinyl, furanyl, furo[2,3-b]pyridinyl, imidazolyl, imidazolidinyl, imidazopyridinyl, isoxazolyl, isothiazolyl, isoquinolinyl, indolyl, indazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazolyl, pyridyl, pyrazolopyrimidinyl, pyrrolizinyl, pyridazyl, pyrazinyl, pyrimidyl, 4-oxo-1,2-dihydro-4H-pyrrolo[3,2,1-ij]-quinolin-4-yl, quinoxalinyl, quinazolinyl, quinolinyl, quinolizinyl, thiophenyl, triazolyl, triazinyl, tetrazolopyrimidinyl, triazolopyrimidinyl, tetrazolyl, thiazolyl, thiazolidinyl, and substituted versions thereof.

As used herein, the term “heterocyclic” refers to a three to seven-membered ring containing one or more heteroatomic moieties selected from S, SO, SO₂, O, N, or N-oxide, optionally substituted with one or more substituents selected from the group which includes substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, and ureido. Such a ring can be saturated or have one or more degrees of unsaturation. Such a ring may be optionally fused to one or more “heterocyclic” ring(s), aryl ring(s), heteroaryl ring(s) or carbocycle ring(s), each having optional substituents.

Examples of “heterocyclic” moieties include, but are not limited to, 1,4-dioxanyl, 1,3-dioxanyl, pyrrolidinyl, pyrrolidin-2-onyl, piperidinyl, imidazolidine-2,4-dionepiperidinyl, piperazinyl, piperazine-2,5-dionyl, morpholinyl, dihydropyranyl, dihydrocinnolinyl, 2,3-dihydrobenzo[1,4]dioxinyl, 3,4-dihydro-2H-benzo[b][1,4]-dioxepinyl, tetrahydropyranyl, 2,3-dihydrofuranyl, 2,3-dihydrobenzofuranyl, dihydroisoxazolyl, tetrahydrobenzodiazepinyl, tetrahydroquinolinyl, tetrahydrofuranyl, tetrahydronaphthyridinyl, tetrahydropurinyl, tetrahydrothiopyranyl, tetrahydrothiophenyl, tetrahydroquinoxalinyl, tetrahydropyridinyl, tetrahydrocarbolinyl, 4H-benzo[1,3]-dioxinyl, benzo[1,3]dioxonyl, 2,2-difluorobenzo-[1,3]-dioxonyl, 2,3-dihydro-phthalazine-1,4-dionyl, and isoindole-1,3-dionyl.

As used herein, the term “alkoxy” refers to the group —ORₐ, where Rₐ is alkyl as defined above. Exemplary alkoxy groups useful in the present invention include, but are not limited to, methoxy, difluoromethoxy, trifluoromethoxy, ethoxy, n-propoxy, isopropoxy, n-butoxy and t-butoxy.

As used herein the term “aralkoxy” refers to the group —ORₐR_{b}, wherein Rₐ is alkyl and R_{b} is aryl as defined above.

As used herein the term “aryloxy” refers to the group —OR_{b}, wherein R_{b} is aryl as defined above.

As used herein, the term “mercapto” refers to the group —SH.

As used herein, the term “sulfanyl” refers to the group —SR_{c}, wherein R_{c} is substituted alkyl, substituted carbocycle, aryl, heteroaryl or heterocyclic, as defined above.

As used herein, the term “sulfinyl” refers to the group —S—(O)R_{c}, wherein R_{c} is substituted alkyl, substituted carbocycle, aryl, heteroaryl or heterocyclic, as defined above.

As used herein, the term “sulfonyl” refers to the group —S(O)₂R_{c}, wherein R_{c} is substituted alkyl, substituted carbocycle, aryl, heteroaryl or heterocyclic, as defined above.

As used herein, the term “oxo” refers to the group ═O.

As used herein, the term “hydroxy” refers to the group —OH.

As used herein, the term “amino” refers to the group —NH₂. The amino group is optionally substituted by substituted alkyl, substituted carbocycle, aryl, heteroaryl or heterocyclic, as defined above.

As used herein, the term “cyano” refers to the group —CN.

As used herein, the term “aminosulfonyl” refers to the group —S(O)₂NH₂. The aminosulfonyl group is optionally substituted by substituted alkyl, substituted carbocycle, aryl, heteroaryl or heterocyclic, as defined above.

As used herein, the term “sulfonylamino” refers to the group —NHS(O)₂R_{c} wherein R_{c} is substituted alkyl, substituted carbocycle, aryl, heteroaryl or heterocyclic, as defined above.

As used herein, the term “carboxyamide” refers to the group —NHC(O)R_{c} wherein R_{c} is substituted alkyl, substituted carbocycle, aryl, heteroaryl or heterocyclic, as defined above.

As used herein, the term “carboxy” refers to the group —C(O)OH. The carboxy group is optionally substituted by substituted alkyl, substituted carbocycle, aryl, heteroaryl or heterocyclic, as defined above.

As used herein, the term “aminocarbonyl” refers to the group —C(O)NH₂. The aminocarbonyl group is optionally substituted by substituted alkyl, substituted carbocycle, aryl, heteroaryl or heterocyclic, as defined above.

As used herein, the term “ureido” refers to the group —NHC(O)NHR_{c} wherein R_{c} is hydrogen, alkyl, carbocycle or aryl as defined above.

As used herein, the term “guanidino” refers to the group —NH C(═NH)NH₂.

As used herein, the term “acyl” refers to the group —C(O)R_{c}, wherein R_{c} is alkyl, carbocycle, or heterocyclic as defined herein.

As used herein, the term “aroyl” refers to the group —C(O)R_{b}, wherein R_{b} is aryl as defined herein.

As used herein, the term “heteroaroyl” refers to the group —C(O)R_{d}, wherein R_{d} is heteroaryl as defined herein.

As used herein, the term “acyloxy” refers to the group —OC(O)R_{c}, wherein R_{c} is alkyl, carbocycle, or heterocyclic as defined herein.

As used herein, the term “aroyloxy” refers to the group —OC(O)R_{b}, wherein R_{b} is aryl as defined herein.

As used herein, the term “heteroaroyloxy” refers to the group —OC(O)R_{d}, wherein R_{d} is heteroaryl as defined herein.

It is to be understood that the present invention also includes a pharmaceutically acceptable salt and an addition salt of the inventive compound, such as a hydrochloride, hydrobromide or trifluoroacetate addition salt and a sodium, potassium and magnesium salt.

The compounds of the present invention may contain one or more asymmetric carbon atoms and may exist in racemic and optically active forms. All of these compounds and diastereomers are incorporated within the scope of the present invention.

One embodiment of the present invention is to provide a compound of formula (Ia) or a pharmaceutically acceptable salt thereof:

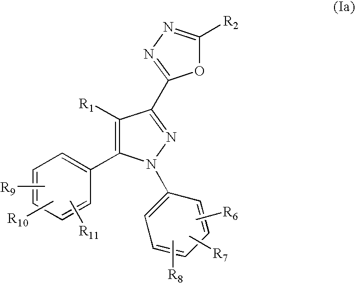

wherein, R₁, R₂, R₆, R₇, R₈, R₉, R₁₀ and R₁₁ have the same meanings as defined above.

Another embodiment of the present invention is provide a compound of formula (Ib) or a pharmaceutically acceptable salt thereof:

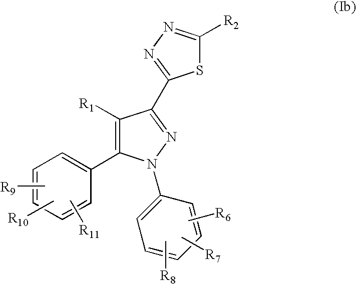

wherein, R₁, R₂, R₆, R₇, R₈, R₉, R₁₀ and R₁₁ have the same meanings as defined above.

A further embodiment of the present invention is provide a compound of formula (Ic) or a pharmaceutically acceptable salt thereof:

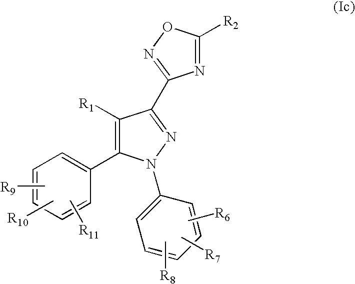

wherein R₁, R₂, R₆, R₇, R₈, R₉, R₁₀ and R₁₁ have the same meanings as defined above.

A still another embodiment of the present invention is to provide a compound of formula (Id) or a pharmaceutically acceptable salt thereof:

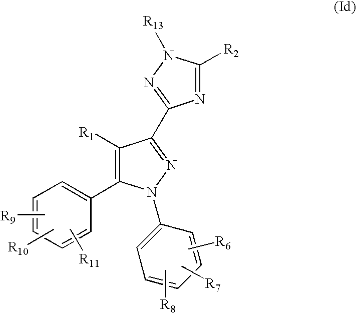

wherein, R₁, R₂, R₆, R₇, R₈, R₉, R₁₀, R₁₁ and R₁₃ have the same meanings as defined above.

The present invention also provides a compound of formula (Ie) or (If) or a pharmaceutically acceptable salt thereof:

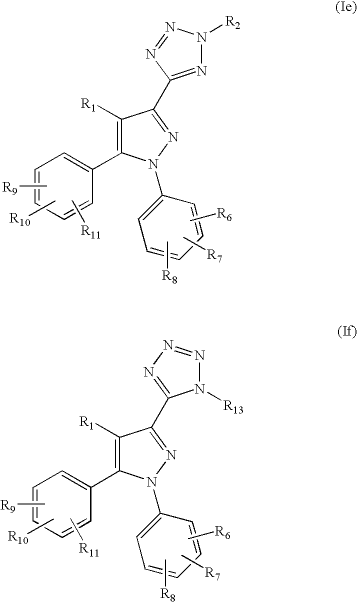

wherein, R₁, R₂, R₆, R₇, R₈, R₉, R₁₀, R₁₁ and R₁₃ have the same meanings as defined above.

The compound of formula (Ia) may be prepared by (i) reacting a carboxylic acid derivative (5) with a hydrazide compound (7) or a semicarbazide compound (12) in the presence of a coupling agent, e.g., EDCI, DMAP, and (ii) cyclizing the resulting product using a dehydrating agent to obtain an 1,3,4-oxadiazole compound of formula (Ia), as shown in Reaction Scheme 1.

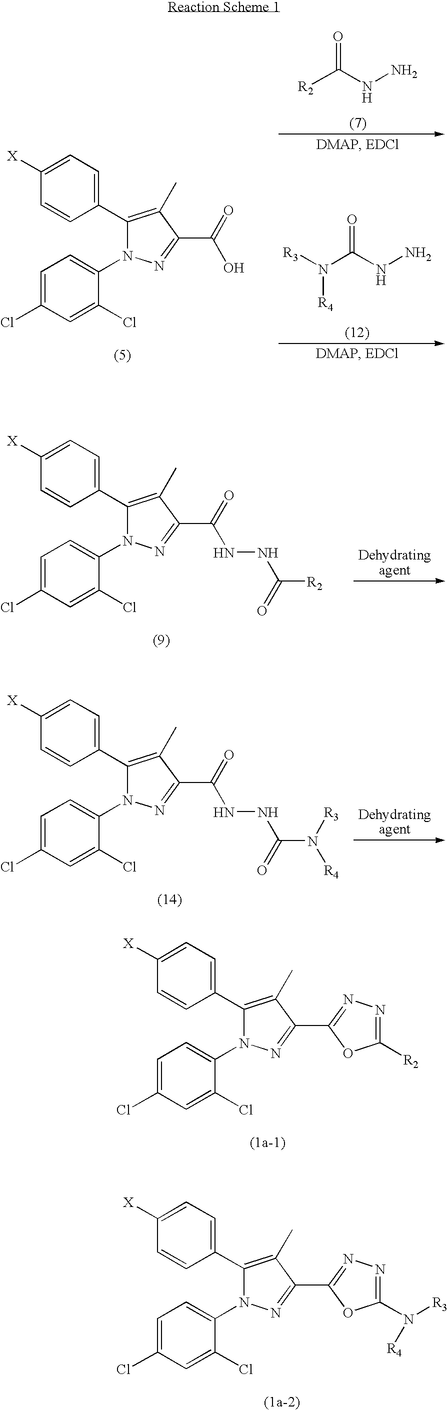

wherein, R₁ to R₄ have the same meanings as defined above.

The cyclization may be conducted using Burgess reagent as a dehydrating agent while applying microwave irradiation thereon (See Leber, J. is D. et al., WO 2005/032550), or using triphenylphosphine with carbon tetrachloride and a base such triethylamine in a suitable solvent such as acetonitrile and THF.

The carboxylic acid derivative (5) used as a starting material in preparing the compound of formula (Ia) may be prepared by a conventional method, e.g., by treating an acetophenone derivative (1) with an organic base such as lithium hexamethyldisilazide (LHMDS) to produce a corresponding alkali metal salt (2), reacting the resulting salt with an equimolar amount of diethyl oxalate to provide a ketoester salt (3), reacting the salt (3) with a hydrazine derivative in refluxing acetic acid to obtain a pyrazole-3-carboxylic ester (4), and transforming the ester (4) into an acid form (5) using an alkaline agent such as potassium hydroxide or lithium hydroxide, followed by acidification (See Barth, F. et al., U.S. Pat. No. 5,462,960), as shown in Reaction Scheme 2.

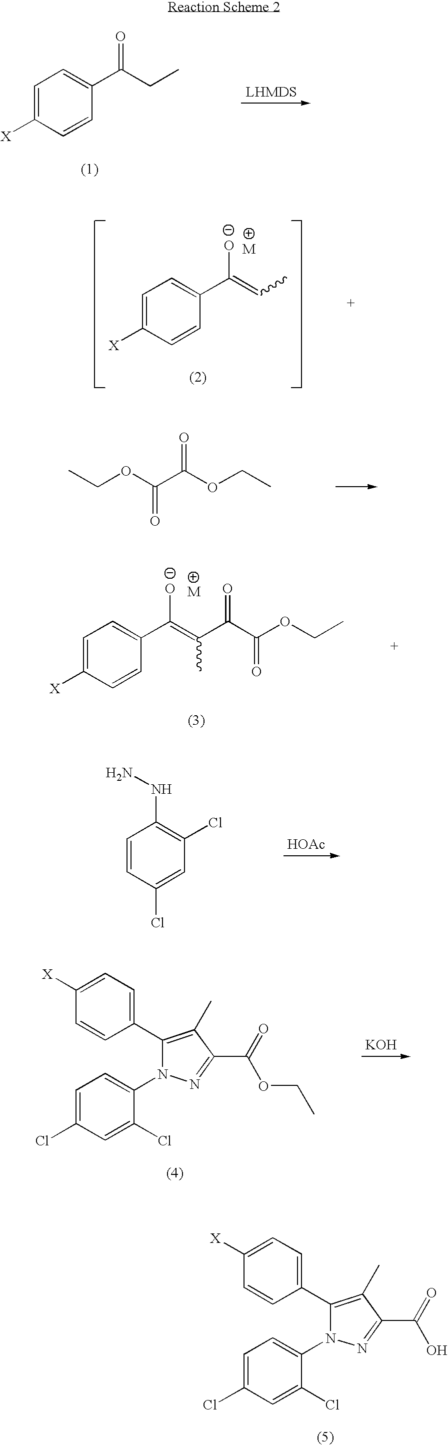

The hydrazide compound (7) which may be used in preparing the compound of formula (Ia) may be prepared by treating an ester or a carboxylic acid with hydrazine, and also, the semicarbazide compound (12) may be prepared by treating carbamyl chloride or isocynate with hydrazine, as shown in Reaction Scheme 3.

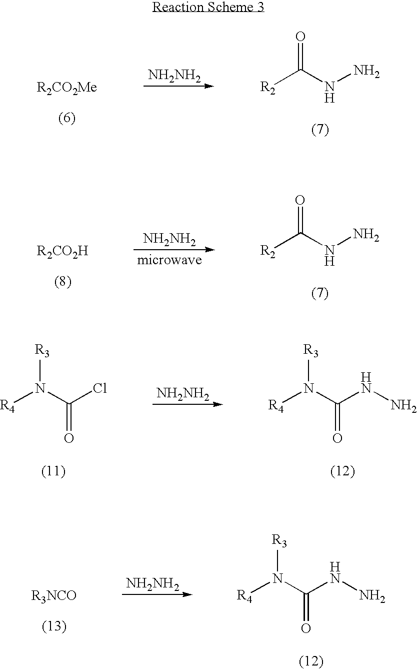

wherein, R₁ to R₄ have the same meanings as defined above.

The compound of formula (Ib) may be prepared by (i) reacting a carboxylic acid derivative (5) with a hydrazide compound (7) in the presence of coupling agents, e.g., EDCI, DMAP, and (ii) cyclizing the resulting product using a Lawesson's reagent, which can be conducted with microwave irradiation (See Kiryanov, A. A., Sampson, P., Seed, A. J., J. Org. Chem. 2001, 665, 7925-7929), as shown in Reaction Scheme 4.

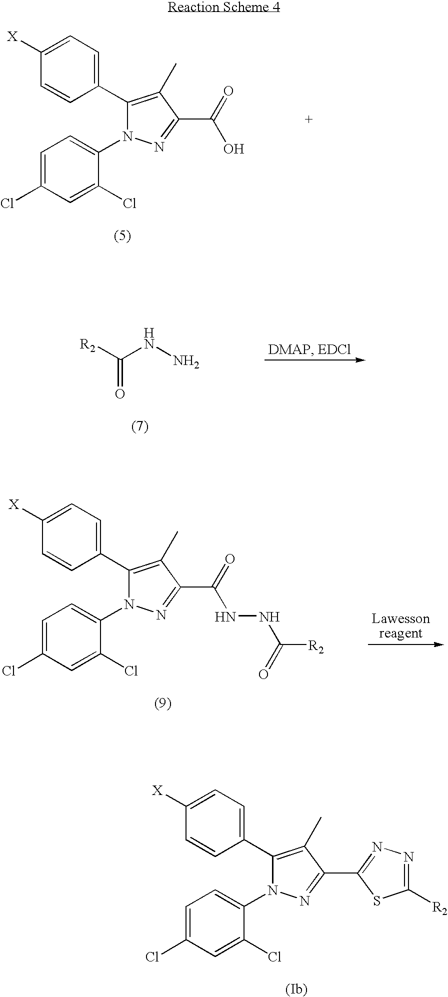

wherein, R₁ and R₂ have the same meanings as defined above.

The compound of formula (Ic) may be prepared by (i) reacting a nitrile intermediate (19) with hydroxylamine in a solvent, e.g., MeOH, (ii) acylating the resulting N-hydroxyamidine (20) with an activated carboxylic acid in the presence of a coupling agent, e.g., DCC, EDCI or CDI, and (iii) cyclizing the acylated compound (21) in the presence of a base by heating, e.g., microwave irradiation, as shown in Reaction Scheme 5.

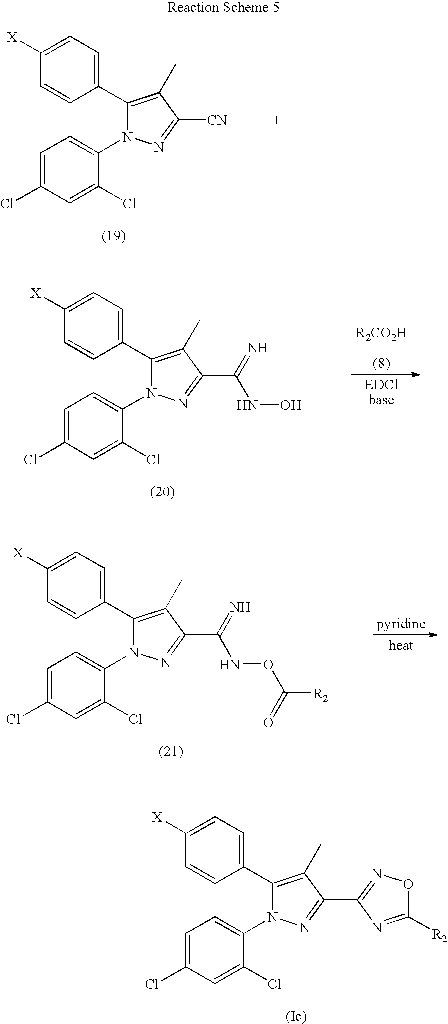

wherein, R₁ and R₂ have the same meanings as defined above.

In Reaction Scheme 5, step (i) may be conducted in heated methyl alcohol. The acylation of step (ii) may be conducted in the presence of a suitable base such as triethylamine or N-methyl morpholine in a solvent such as methylene chloride, THF or acetonitrile. The cyclization step (iii) may be conducted in a solvent such as acetonitrile or THF, and exemplary bases which may be used in this step include pyridine, N,N-diisopropylethylamine or tetrabutylammonium fluoride. Also, the process of isolating N-acyloxyamidine (21) may be omitted, in case N-hydroxyamidine (20) was converted to 1,2,4-oxadiazole (22) in a continuous process (See, Colandrea, V. J. et al., WO 2005/058848).

The nitrile intermediate (19) used in preparing the compound of formula (Ic) may be prepared by [3+2] cycloaddition reaction disclosed in J. Med. Chem. 1999, 42, 769-776, as shown in Reaction Scheme 6.

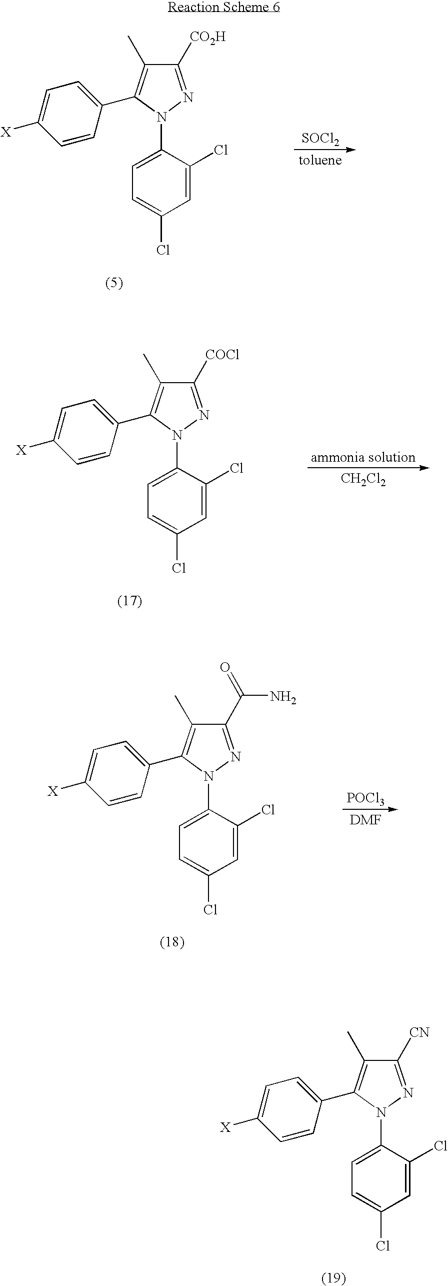

The compound of formula (Id) may be prepared by reacting a nitrile intermediate (19) with a hydrazide compound (7) in the presence of a catalyst such as potassium carbonate in a suitable solvent such as 1-butanol under a reflux condition, to obtain a triazole, as shown in Reaction Scheme 7.

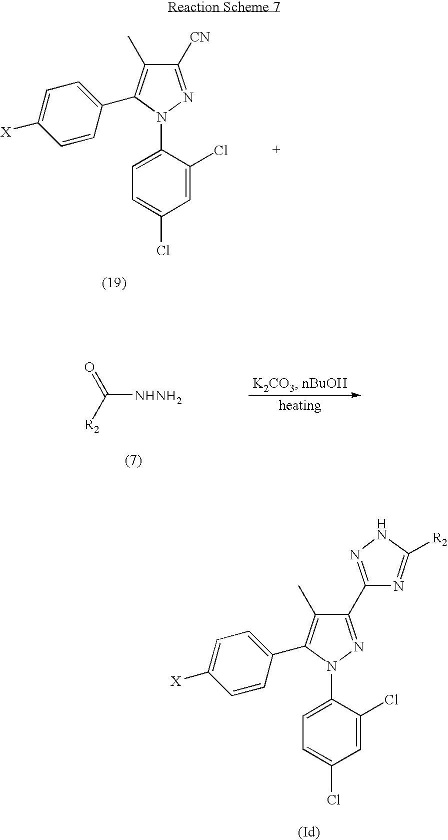

wherein, R₂ has the same meaning as defined above.

The compound of formula (Ie) or (If) may be prepared by reacting a nitrile intermediate (19) with sodium azide in the presence of a base (e.g., ammonium chloride) in a solvent (e.g., DMF) with microwave irradiation, according to [3+2] cycloaddition reaction, to obtain a tetrazole, which may be alkylated by a reaction with an alkyl halide in the presence of potassium carbonate in DMF to obtain alkyl tetrazoles, as shown in Reaction Scheme 8.

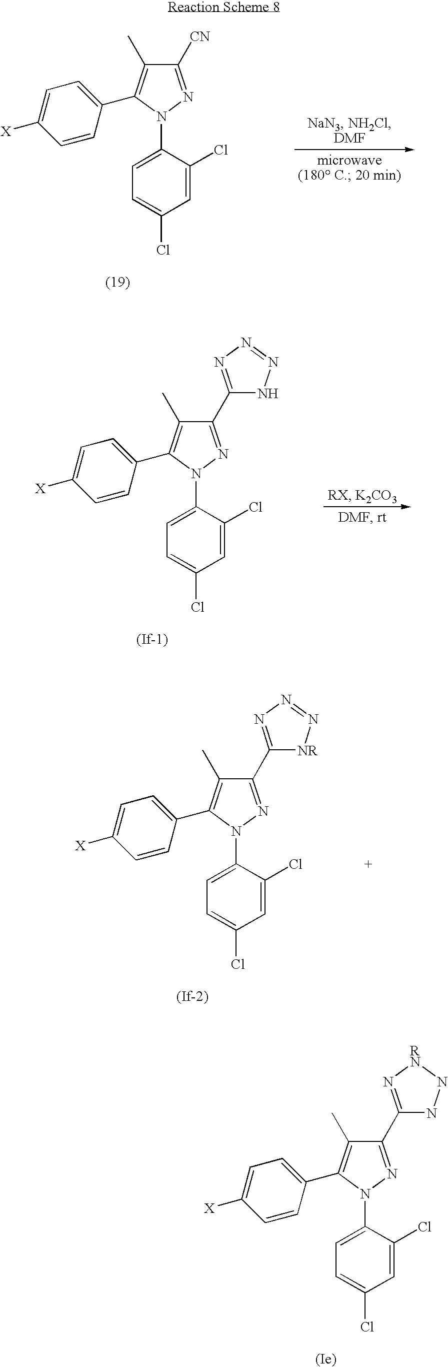

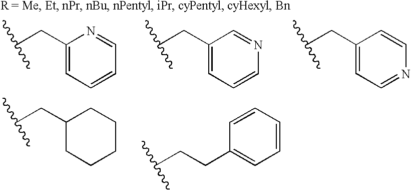

In Reaction Scheme 8, when RX is a primary alkyl halide, the alkylation can be conducted at room temperature. However, when a secondary alkyl halide is used, the reaction temperature is preferably conducted at 80□.

Also, the alkylation of the tetrazole may be conducted using an aliphatic alcohol in the presence of diisopropylazodicarboxylate (DIAD) and triphenylphophine (PPh₃) in THF at 0□, as shown in Reaction Scheme 9.

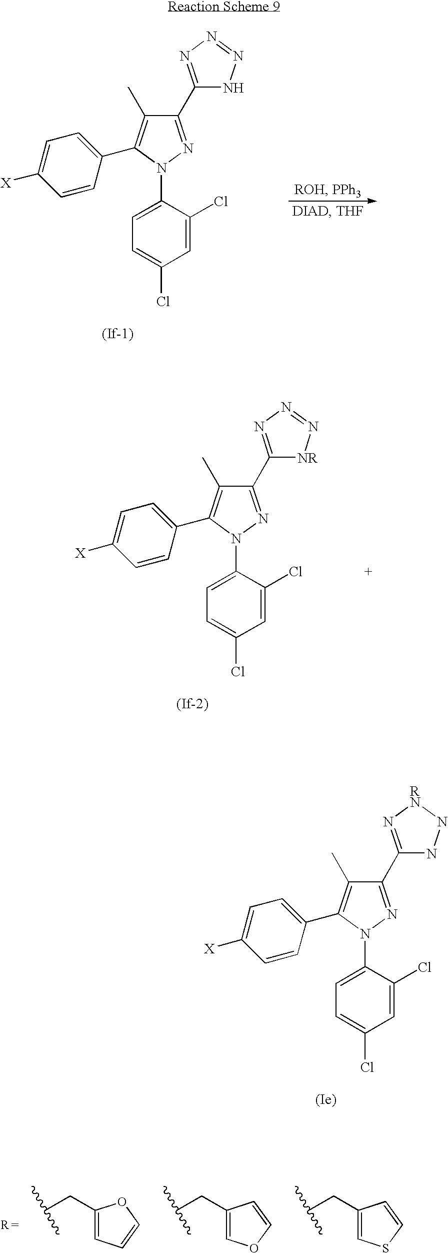

Alternatively, the compound of formula (If) may be prepared by treating acyl chloride (17) with an amine in the presence of triethylamine in methylene chloride to produce an amide (26) and reacting the resulting amide with hydrazoic acid in the presence of phosphorus pentachloride in toluene as shown in Reaction Scheme 10.

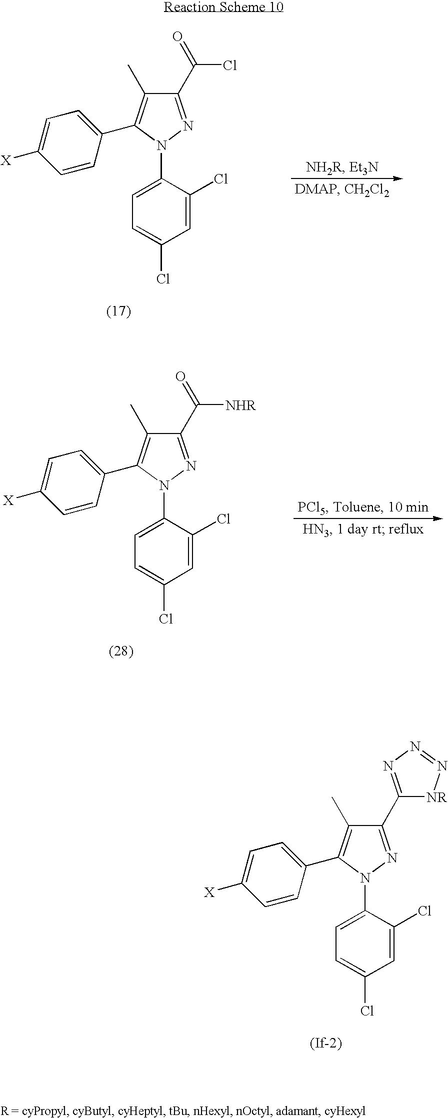

The inventive heteroaryl-pyrazole compound of formula (I) is effective as a cannabinoid CB₁ receptor inverse agonist or antagonist, thereby preventing or treating obesity and obesity-related metabolic disorders.

Accordingly, the present invention provides a pharmaceutical composition for preventing or treating obesity and obesity-related metabolic disorders, which comprises the compound of formula (I) or a pharmaceutically acceptable salt thereof as an active ingredient and a pharmaceutically acceptable carrier.

Further, the present invention provides a method for preventing or treating obesity and obesity-related metabolic disorders in a mammal, which comprises administering the compound of formula (I) of claim 1 to the mammal.

Also, the present invention provides a method for inhibiting cannabinoid CB₁ receptor in a mammal, which comprises administering the compound of formula (I) of claim 1 to the mammal.

As used herein, the term “obesity-related metabolic disorders” refers to chronic diseases that require treatment to reduce the excessive health risks associated with obesity and exemplary disorders include type 2 diabetes mellitus, cardiovascular and hypertension, hyperlipidaemia, fibrinolytic abnormalities.

The pharmaceutical composition may be administered orally, intramuscularly or subcutaneously. The formulation for oral administration may take various forms such as a syrup, tablet, capsule, cream and lozenge. A syrup formulation will generally contain a suspension or solution of the compound or its salt in a liquid carrier, e.g., ethanol, peanut oil, olive oil, glycerine or water, optionally with a flavoring or coloring agent. When the composition is in the form of a tablet, any one of pharmaceutical carriers routinely used for preparing solid formulations may be used. Examples of such carriers include magnesium stearate, terra alba, talc, gelatin, acacia, stearic acid, starch, lactose and sucrose. When the composition is in the form of a capsule, any of the routine encapsulation procedures may be employed, e.g., using the aforementioned carriers in a hard gelatin capsule shell. When the composition is formulated in the form of a soft gelatin shell capsule, any of the pharmaceutical carrier routinely used for preparing dispersions or suspensions may be prepared using an aqueous gum, cellulose, silicate or oil. The formulation for intramuscular or subcutaneous administration may take a liquid form such as a solution, suspension and emulsion which includes aqueous solvents such as water, physiological saline and Ringer's solution; or lipophilic solvents such as fatty oil, sesame oil, corn oil and synthetic fatty acid ester.

Preferably the composition is formulated in a specific dosage form for a particular patient.

Each dosage unit for oral administration contains suitably from 0.1 mg to 500 mg/Kg, and preferably from 1 mg to 100 mg/Kg of the compound of Formula (I) or its pharmaceutically acceptable salt.

The suitable daily dosage for oral administration is about 0.01 mg/Kg to 40 mg/Kg of the compound of Formula (I) or its pharmaceutically acceptable salt, may be administered 1 to 6 times a day, depending on the patient's condition.

The present invention is further described and illustrated in Examples provided below, which are, however, not intended to limit the scope of the present invention.

### EXAMPLE

As used herein the symbols and conventions used describing the processes, schemes and examples of the present invention are consistent with those used in the contemporary scientific literature, for example, the Journal of the American Chemical Society or the Journal of Biological Chemistry. Unless otherwise noted, all starting materials were obtained from commercial suppliers and used without further purification.
Hz (Hertz)
Tr (retention time)
MeOH (methanol)
TFA (trifluoroacetic acid)
EtOH (ethanol)
DMSO (dimethylsulfoxide)
DCM (dichlromethane)
DMY (N,N-dimethylformamide)
CDI (1,1-carbnyldiimidazole)
HOSu (N-hydroxysuccinimide)
HOBT (1-hydroxybenzotriazole)
Boc (tert-butyloxycarbonyl)
mCPBA (meta-chloroperbenzoic acid)
FMOC (9-fluorenylmethoxycarbonyl)
DCC (dicyclohexylcarbodiimide)
Cbz (benzyloxycarbonyl)
NMM (N-methyl morpholine)
HOAt (1-hydroxy-7-azabenzotriazole)
TBAF (tetra-n-butylammonium fluoride)
THP (tetrahydro-2H-pyran-2-yl)
DMAP (4-dimethylaminopyridine)
HPLC (high pressure liquid chromatography)
BOP (bis(2-oxo-3-oxazolidinyl)phosphinic chloride);
EDCI (1-ethyl-3-[3-dimethylaminopropyl]carbodiimide hydrochloride)
HBTU (O-Benzotriazolel-yl-N,N,N′,N′-tetramethyluronium hexafluorophosphate)
TLC (thin layer chromaography)
RP (reverse phase)
i-PrOH (isopropanol)
TEA (triethylamine)
THF (tetrahyrdofuran)
EtOAc (ethyl acetate)
HOAc (acetic acid)
Ac (acetyl)
Bn (benzyl)

All references to ether are to diethyl ether; brine refers to a saturated aqueous solution of NaCl. Unless otherwise indicated, all temperatures are expressed in□ (degrees Centigrade). All reactions are conducted under an inert atmosphere at room temperature unless otherwise noted, and all solvents are of the highest available purity unless otherwise indicated.

Microwave reaction was conducted with a Biotage microwave reactor.

¹H NMR spectra were recorded on either a Jeol ECX-400, or a Jeol JNM-LA300 spectrometer. Chemical shifts were expressed in parts per million (ppm, δ units). Coupling constants are in units of hertz (Hz). Splitting patterns describe apparent multiplicities and are designated as s (singlet), d(doublet), t (triplet), q (quartet), quint (quintet), m (multiplet), br (broad).

Mass spectra were obtained with either a Micromass, Quattro LC Triple Quadruple Tandem Mass Spectometer, ESI or Agilent, 1100LC/MSD, ESI.

For preparative HPLC, ca 100 mg of a product was injected in 1 mL of DMSO onto a SunFire™ Prep C18 OBD 5 um 19×100 mm Column with a 10 min gradient from 10% CH₃CN to 90% CH₃CN in H₂O. Flash chromatography was carried using Merck silica gel 60 (230-400 mesh). Most of the reactions were monitored by thin-layer chromatography on 0.25 mm E. Merck silica gel plates (60F-254), visualized with UV light using a 5% ethanolic phosphomolybdic acid or p-anisaldehyde solution.

The following synthetic schemes are merely illustrative of the methods by which the compounds of the invention may be prepared and are not intended to limit the scope of the invention as defined in the appended claims.

### Preparation of 1,3,4-oxadiazole (Formula (Ia))

### Example 1

### 2-(5-(4-chlorophenyl)-1-(2,4-dichlorophenyl)-4-methyl-1H-pyrazol-3-yl)-5-propyl-1,3,4-oxadiazole

### Step 1: N-butanoyl-N′-[5-(4-Chlorophenyl)-1-(2,4-dichlorophenyl)-4-methyl-1H-pyrazole-3-carbonyl]-hydrazine

Added to a solution of 5-(4-chlorophenyl)-1-(2,4-dichlorophenyl)-4-methyl-1H-pyrazole-3-carboxylic acid (0.40 g, 1.05 mmol), N-butanoyl-hydrazine (0.11 g, 1.05 mmol) and EDCI (0.24 g, 1.26 mmol) dissolved in DCM (11 ml), was DMAP (0.15 g, 1.26 mmol) in one portion at room temperature. The reaction mixture was stirred at room temperature for 6 hr, and then treated with 10% aq. HCl. The organic layer was collected, and evaporated under a vacuum. The crude mixture was further purified by preparative HPLC, to obtain 0.38 g (0.81 mmol, 77%) of the title compound as a yellow solid.

¹H NMR (400 MHz, CDCl₃)) [symbol] 7.40 (br s, 1H), 7.31-7.27 (m, 4H), 7.08-7.03 (m, 2H), 2.33 (s, 3H), 2.31 (t, J=7.8 Hz, 2H), 1.72 (m, 2H), 0.97 (t, J=7.3 Hz, 3H).

MH+ 463.

### Step 2: 2-(5-(4-chlorophenyl)-1-(2,4-dichlorophenyl)-4-methyl-1H-pyrazol-3-yl)-5-propyl-1,3,4-oxadiazole

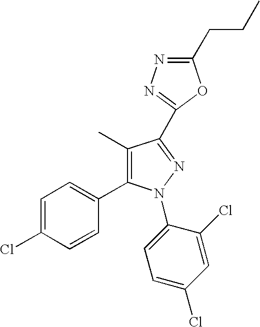

N-butanoyl-N′-[5-(4-Chlorophenyl)-1-(2,4-dichlorophenyl)-4-methyl-1H-pyrazole-3-carbonyl]-hydrazine (0.35 g, 0.75 mmol) obtained in Step 1 was added to a microwave reactor containing Burgess reagent (0.45 g, 1.88 mmol) in THF (2 mL). The capped reactor was placed in a microwave reactor and the mixture was irradiated at 140□ for 15 min. The reaction product was purified by preparative HPLC to provide the title compound (0.21 g, 0.46 mmol, 61%) as a yellow solid.

¹H NMR (400 MHz, CDCl₃) [symbol] 7.41 (d, J=2.3 Hz, 1H), 7.34-7.28 (m, 4H), 7.09-7.13 (m, 2H), 2.90 (t, J=7.6 Hz, 2H), 2.45 (s, 3H), 1.88 (m, 2H), 1.03 (t, J=7.6 Hz, 3H).

MH+ 447.

The following compounds of Examples 2 to 21 were obtained by repeating the procedure of Example 1.

### Example 2

### 2-(5-(4-chlorophenyl)-1-(2,4-dichlorophenyl)-4-methyl-1H-pyrazol-3-yl)-5-(pyridin-4-yl)-1,3,4-oxadiazole

¹H NMR (400 MHz, CDCl₃) δ 8.78 (d, J=7.43 Hz, 1H), 8.01 (d, J=5.04 Hz, 1H), 7.41 (d, J=1.84 Hz, 1H), 7.37-7.29 (m, 4H), 7.11 (d, J=8.72 Hz, 2H), 2.48 (s, 3H).

MH+ 482.

### Example 3

### 2-(5-(4-chlorophenyl)-1-(2,4-dichlorophenyl)-4-methyl-1H-pyrazol-3-yl)-5-methyl-1,3,4-oxadiazole

¹H NMR (400 MHz, CDCl₃) δ 7.43 (d, J=1.84 Hz, 1H), 7.37-7.29 (m, 4H), 7.11 (dt, J=2.28, 8.24 Hz, 2H), 2.63 (s, 3H), 2.46 (s, 3H).

MH+ 419.

### Example 4

### 2-Butyl-5-(5-(4-chlorophenyl)-1-(2,4-dichlorophenyl)-4-methyl-1H-pyrazol-3-yl)-1,3,4-oxadiazole

¹H NMR (400 MHz, CDCl₃) δ 7.43 (d, J=1.84 Hz, 1H), 7.37-7.29 (m, 4H), 7.12 (dt, J=2.28, 8.24 Hz, 2H), 2.94 (t, J=7.56 Hz, 2H), 2.46 (s, 3H), 1.85 (m, 2H), 1.45 (m, 2H), 0.96 (t, J=7.36 Hz, 3H).

MH+ 461.

### Example 5

### 2-(5-(4-chlorophenyl)-1-(2,4-dichlorophenyl)-4-methyl-1H-pyrazol-3-yl)-5-cyclopentyl-1,3,4-oxadiazole

¹H NMR (400 MHz, CDCl₃) δ 7.41 (d, J=1.84 Hz, 1H), 7.37-7.29 (m, 4H), 7.11 (dt, J=2.28, 8.24 Hz, 2H), 3.39 (m, 1H), 2.45 (s, 3H), 2.14 (m, 2H), 2.04 (m, 2H), 1.84 (m, 2H), 1.71 (m, 2H).

MH+ 473.

### Example 6

### 2-(5-(4-chlorophenyl)-1-(2,4-dichlorophenyl)-4-methyl-1H-pyrazol-3-yl)-5-cyclohexyl-1,3,4-oxadiazole

¹H NMR (400 MHz, CDCl₃) δ 7.41 (d, J=1.84 Hz, 1H), 7.37-7.29 (m, 4H), 7.11 (dt, J=2.28, 8.24 Hz, 2H), 3.00 (m, 1H), 2.44 (s, 3H), 2.16-2.12 (m, 2H), 1.87-1.81 (m, 2H), 1.78-1.65 (m, 3H), 1.44-1.28 (m, 3H).

MH+ 487.

### Example 7

### 2-(5-(4-chlorophenyl)-1-(2,4-dichlorophenyl)-4-methyl-1H-pyrazol-3-yl)-5-cyclopropyl-1,3,4-oxadiazole

MH+ 445.

### Example 8

### 2-(5-(4-chlorophenyl)-1-(2,4-dichlorophenyl)-4-methyl-1H-pyrazol-3-yl)-5-(furan-2-yl)-1,3,4-oxadiazole

MH+ 471.

### Example 9

### 2-(5-(4-chlorophenyl)-1-(2,4-dichlorophenyl)-4-methyl-1H-pyrazol-3-yl)-5-cyclobutyl-1,3,4-oxadiazole

¹H NMR (400 MHz, CDCl₃) [symbol] 7.43 (d, J=2.3 Hz, 1H), 7.34-7.28 (m, 4H), 7.13-7.09 (m, 2H), 4.01 (m, 1H), 2.62-2.52 (m, 2H), 2.49 (s, 3H), 2.47-2.39(m, 2H), 2.20-2.00 (m, 2H).

MH+ 459.

### Example 10

### 2-(5-(4-chlorophenyl)-1-(2,4-dichlorophenyl)-4-methyl-1H-pyrazol-3-yl)-5-(pyrazin-2-yl)-1,3,4-oxadiazole

¹H NMR (400 MHz, CDCl₃) δ 9.53 (s, 1H), 8.76 (m, 1H), 7.43 (d, J=1.84 Hz, 1H), 7.39 (d, J=8.68 Hz, 1H), 7.36-7.30 (m, 4H), 7.15 (d, J=8.24 Hz, 2H), 2.55 (s, 3H).

MH+ 483.

### Example 11

### 2-(5-(4-chlorophenyl)-1-(2,4-dichlorophenyl)-4-methyl-1H-pyrazol-3-yl)-5-(thiophen-2-yl)-1,3,4-oxadiazole

MH+ 487.

### Example 12

### 2-(5-(4-chlorophenyl)-1-(2,4-dichlorophenyl)-4-methyl-1H-pyrazol-3-yl)-5-(pyridin-2-ylmethyl)-1,3,4-oxadiazole

¹H NMR (400 MHz, CDCl₃) δ 8.55 (d, J=4.12 Hz, 1H), 7.66 (dt, J=1.84, 7.80 Hz, 1H), 7.41 (d, J=1.84 Hz, 1H), 7.35-7.29 (m, 5H), 7.20 (dd, J=5.04, 7.32 Hz, 1H), 7.13-7.09 (m, 2H), 4.52 (s, 2H), 2.45 (s, 3H).

MH+ 496.

### Example 13

### 2-(5-(4-chlorophenyl)-1-(2,4-dichlorophenyl)-4-methyl-1H-pyrazol-3-yl)-5-isopropyl-1,3,4-oxadiazole

¹H NMR (400 MHz, CDCl₃) δ 7.41 (d, J=2.28 Hz, 1H), 7.38-7.29 (m, 4H), 7.13-7.09 (m, 2H), 3.28 (m, 1H), 2.44 (s, 3H), 1.46 (d, J=6.88 Hz, 6H).

MH+ 447.

### Example 14

### 2-(5-(4-chlorophenyl)-1-(2,4-dichlorophenyl)-4-methyl-1H-pyrazol-3-yl)-5-(pentan-3-yl)-1,3,4-oxadiazole

¹H NMR (400 MHz, CDCl₃) δ 7.42 (d, J=2.28 Hz, 1H), 7.38-7.30 (m, 4H), 7.13-7.10 (m, 2H), 2.96 (m, 1H), 2.46 (s, 3H), 1.93-1.79 (m, 2H), 0.94 (t, J=7.32 Hz, 3H).

MH+ 475.

### Example 15

### 2-Benzyl-5-(5-(4-chlorophenyl)-1-(2,4-dichlorophenyl)-4-methyl-1H-pyrazol-3-yl)-1,3,4-oxadiazole

¹H NMR (400 MHz, CDCl₃) δ 7.41 (d, J=1.84 Hz, 1H), 7.38-7.27 (m, 9H), 7.12-7.08 (m, 2H), 4.29 (s, 2H), 2.43 (s, 3H).

MH+ 495.

### Example 16

### 2-(5-(4-Chlorophenyl)-1-(2,4-dichlorophenyl)-4-methyl-1H-pyrazol-3-yl)-5-phenyl-1,3,4-oxadiazole

¹H NMR (300 MHz, CDCl₃) δ 8.22-8.18 (m, 2H), 7.57-7.49 (m, 3H), 7.45-7.44 (m, 1H), 7.41-7.32 (m, 4H), 7.16-7.12 (m, 2H), 2.51 (s, 3H).

MH+ 483.

### Example 17

### 2-(4-Chlorophenyl)-5-(5-(4-chlorophenyl)-1-(2,4-dichlorophenyl)-4-methyl-1H-pyrazol-3-yl)-1,3,4-oxadiazole

¹H-NMR (300 MHz, CDCl₃) δ 8.16-8.12 (m, 2H), 7.52-7.48 (m, 2H), 7.45-7.44 (m, 1H), 7.37-7.32 (m, 4H), 7.16-7.12 (m, 2H), 2.50 (s, 3H).

MH+ 517.

### Example 18

### 2-(Benzofuran-2-yl)-5-(5-(4-chlorophenyl)-1-(2,4-dichlorophenyl)-4-methyl-1H-pyrazol-3-yl)-1,3,4-oxadiazole

¹H-NMR (400 MHz, CDCl₃) δ \ddot{□} [symbol] d, J=7.7 Hz, 1H), 7.64-7.62 (m, 2H), 7.47-7.42 (m, 2H), 7.40-7.38 (m, 1H), 7.36-7.30 (m, 4H), 7.16-7.13 (m, 2H), 2.53 (s, 3H).

MH+ 521.

### Example 19

### 2-(5-(4-Chlorophenyl)-1-(2,4-dichlorophenyl)-4-methyl-1H-pyrazol-3-yl)-5-pentyl-1,3,4-oxadiazole

¹H-NMR (400 MHz, CDCl₃) δ 7.43-7.42 (m, 1H), 7.37-7.30 (m, 4H), 7.13-7.10 (m, 2H), 2.93 (t, J=7.8 Hz, 2H), 2.46 (s, 3H), 1.90-1.83 (m, 2H), 1.45-1.32 (m, 4H), 0.91 (t, J=7.3 Hz, 3H).

MH+ 475.

### Example 20

### 2-(5-(4-Chlorophenyl)-1-(2,4-dichlorophenyl)-4-methyl-1H-pyrazol-3-yl)-5-cycloheptyl-1,3,4-oxadiazole

¹H-NMR (400 MHz, CDCl₃) δ 7.43-7.41 (m, 1H), 7.37-7.29 (m, 4H), 7.13-7.10 (m, 2H), 3.23-3.16 (m, 1H), 2.45 (s, 3H), 2.21-2.14 (m, 4H), 1.98-1.89 (m, 2H), 1.85-1.79 (m, 2H), 1.68-1.53 (m, 4H).

MH+ 501.

### Example 21

### 2-(5-(4-Chlorophenyl)-1-(2,4-dichlorophenyl)-4-methyl-1H-pyrazol-3-yl)-5-(2,4-dichlorophenyl)-1,3,4-oxadiazole

¹H-NMR (400 MHz, CDCl₃) δ 8.04 (d, J=8.4 Hz, 1H), 7.60 (d, J=1.8 Hz, 1H), 7.44 (d, J=2.2 Hz, 1H), 7.41 (d, J=2.2 Hz, 1H), 7.39-7.31 (m, 4H), 7.15-7.12 (m, 2H), 2.51 (s, 3H).

MH+ 548.

### Preparation of 1,3,4-thiadiazole (Formula (Ib))

### Example 22

### 2-(5-(4-chlorophenyl)-1-(2,4-dichlorophenyl)-4-methyl-1H-pyrazol-3-yl)-5-cyclobutyl-1,3,4-thiadiazole

N-cyclobutanoyl-N′-[5-(4-Chlorophenyl)-1-(2,4-dichlorophenyl)-4-methyl-1H-pyrazole-3-carbonyl]-hydrazine (0.14 g, 0.29 mmol) was added to a microwave reactor containing Lawesson's reagent (0.18 g, 0.44 mmol) in 1,4-dioxane (3 mL). The capped reactor was placed into a microwave reactor and the mixture was heated at 180□ for 15 min, and the process was repeated one more time. The reaction mixture was then purified by preparative HPLC to provide the title compound (60 mg, 0.16 mmol, 43%) as a yellow solid.

¹H NMR (400 MHz, CDCl₃) δ 7.43 (d, J=2.3 Hz, 1H), 7.34-7.28 (m, 4H), 7.13-7.09 (m, 2H), 4.01 (m, 1H), 2.62-2.52 (m, 2H), 2.49 (s, 3H), 2.47-2.39 (m, 2H), 2.20-2.00 (m, 2H).

MH+ 475.

The following compounds of Examples 23 to 40 were obtained by repeating the procedure of Example 22.

### Example 23

### 2-(5-(4-chlorophenyl)-1-(2,4-dichlorophenyl)-4-methyl-1H-pyrazol-3-yl)-5-cyclopentyl-1,3,4-thiadiazole

¹H NMR (400 MHz, CDCl₃) δ 7.42 (d, J=1.84 Hz, 1H), 7.34-7.28 (m, 4H), 7.11 (dt, J=2.28, 8.24 Hz, 2H), 3.60 (m, 1H), 2.49 (s, 3H), 2.26 (m, 2H), 1.94-1.81 (m, 4H), 1.74 (m, 2H).

MH+ 489.

### Example 24

### 2-(5-(4-chlorophenyl)-1-(2,4-dichlorophenyl)-4-methyl-1H-pyrazol-3-yl)-5-cyclohexyl-1,3,4-thiadiazole

¹H NMR (400 MHz, CDCl₃) δ 7.41 (d, J=1.84 Hz, 1H), 7.37-7.29 (m, 4H), 7.11 (dt, J=2.28, 8.24 Hz, 2H), 2.99 (m, 1H), 2.44 (s, 3H), 2.16-2.12 (m, 2H), 1.87-1.81 (m, 2H), 1.78-1.65 (m, 3H), 1.44-1.28 (m, 3H).

MH+ 487.

### Example 25

### 2-(5-(4-chlorophenyl)-1-(2,4-dichlorophenyl)-4-methyl-1H-pyrazol-3-yl)-5-cyclopropyl-1,3,4-thiadiazole

MH+ 461.

### Example 26

### 2-(5-(4-chlorophenyl)-1-(2,4-dichlorophenyl)-4-methyl-1H-pyrazol-3-yl)-5-(furan-2-yl)-1,3,4-thiadiazole

MH+ 486.

### Example 27

### 2-(5-(4-chlorophenyl)-1-(2,4-dichlorophenyl)-4-methyl-1H-pyrazol-3-yl)-5-(pyrazin-2-yl)-1,3,4-thiadiazole

¹H-NMR (400 MHz, CDCl₃) δ 9.64 (d, J=1.40 Hz, 1H), 8.67 (d, J=2.28 Hz, 1H), 8.64 (dd, J=1.40, 2.32 Hz, 1H), 7.44 (d, J=1.84 Hz, 1H), 7.36-7.29 (m, 4H), 7.15-7.11 (m, 2H), 2.56 (s, 3H).

MH+ 499.

### Example 28

### 2-(5-(4-chlorophenyl)-1-(2,4-dichlorophenyl)-4-methyl-1H-pyrazol-3-yl)-5-(thiophen-2-yl)-1,3,4-thiadiazole

¹H-NMR (400 MHz, CDCl₃) δ 7.58 (dd, J=0.92, 3.68 Hz, 1H), 7.50 (dd, J=1.36, 5.04 Hz, 1H), 7.44 (d, J=1.84 Hz, 1H), 7.36-7.29 (m, 4H), 7.15-7.11 (m, 3H), 2.52 (s, 3H).

MH+ 503.

### Example 29

### 2-(5-(4-chlorophenyl)-1-(2,4-dichlorophenyl)-4-methyl-1H-pyrazol-3-yl)-5-(pyridin-2-ylmethyl)-1,3,4-thiadiazole

¹H-NMR (400 MHz, CDCl₃) δ 8.59 (d, J=5.04 Hz, 1H), 7.66 (dt, J=1.84, 7.76 Hz, 1H), 7.41 (d, J=1.84 Hz, 1H), 7.36 (d, J=7.76 Hz, 1H), 7.33-7.28 (m, 4H), 7.20 (dd, J=5.04, 7.32 Hz, 1H), 7.13-7.09 (m, 2H), 4.66 (s, 2H), 2.48 (s, 3H).

MH+ 512.

### Example 30

### 2-(5-(4-chlorophenyl)-1-(2,4-dichlorophenyl)-4-methyl-1H-pyrazol-3-yl)-5-isopropyl-1,3,4-thiadiazole

¹H-NMR (400 MHz, CDCl₃) δ 7.42 (d, J=1.84 Hz, 1H), 7.33-7.28 (m, 3H), 7.13-7.09 (m, 2H), 3.50 (m, 1H), 2.49 (s, 3H), 1.47 (d, J=6.88 Hz, 6H). MH+ 463.

### Example 31

### 2-benzyl-5-(5-(4-chlorophenyl)-1-(2,4-dichlorophenyl)-4-methyl-1H-pyrazol-3-yl)-1,3,4-thiadiazole

¹H-NMR (400 MHz, CDCl₃) δ 7.40 (br s, 1H), 7.34-7.28 (m, 9H), 7.16-7.12 (m, 2H), 4.45 (s, 2H), 2.48 (s, 3H).

MH+ 511.

### Example 32

### 2-Butyl-5-(5-(4-chlorophenyl)-1-(2,4-dichlorophenyl)-4-methyl-1H-pyrazol-3-yl)-1,3,4-thiadiazole

¹H-NMR (400 MHz, CDCl₃) δ 7.43-7.42 (m, 1H), 7.34-7.29 (m, 4H), 7.13-7.10 (m, 2H), 3.14 (t, J=7.8 Hz, 2H), 2.49 (s, 3H), 1.85-1.79 (m, 2H), 1.52-1.42 (m, 2H), 0.97 (t, J=7.3 Hz, 3H).

MH+ 477.

### Example 33

### 2-(5-(4-Chlorophenyl)-1-(2,4-dichlorophenyl)-4-methyl-1H-pyrazol-3-yl)-5-phenyl-1,3,4-thiadiazole

¹H-NMR (300 MHz, CDCl₃) δ 8.05-8.01 (m, 2H), 7.51-7.48 (m, 3H), 7.45-7.44 (m, 1H), 7.35-7.31 (m, 4H), 7.16-7.12 (m, 2H), 2.55 (s, 3H). MH+ 497.

### Example 34

### 2-(5-(4-Chlorophenyl)-1-(2,4-dichlorophenyl)-4-methyl-1H-pyrazol-3-yl)-5-(cyclopentylmethyl)-1,3,4-thiadiazole

¹H-NMR (300 MHz, CDCl₃) δ 7.43-7.42 (m, 1H), 7.35-7.28 (m, 4H), 7.13-7.10 (m, 2H), 3.14 (d, J=7.5, 2H), 2.50 (s, 3H), 2.36-2.26 (m, 1H), 1.88-1.84 (m, 2H), 1.71-1.54 (m, 4H), 1.37-1.25 (m, 2H).

MH+ 503.

### Example 35

### 2-(5-(4-Chlorophenyl)-1-(2,4-dichlorophenyl)-4-methyl-1H-pyrazol-3-yl)-5-(tetrahydro-2H-pyran-4-yl)-1,3,4-thiadiazole

¹H-NMR (300 MHz, CDCl₃) δ 7.43-7.42 (m, 1H), 7.35-7.30 (m, 4H), 7.14-7.10 (m, 2H), 4.11-4.07 (m, 2H), 3.63-3.55 (m, 2H), 3.50-3.40 (m, 1H), 2.50 (s, 3H), 2.14-2.11 (m, 2H), 2.05-1.92(m, 2H).

MH+ 505

### Example 36

### 2-(5-(4-Chlorophenyl)-1-(2,4-dichlorophenyl)-4-methyl-1H-pyrazol-3-yl)-5-(tetrahydrofuran-2-yl)-1,3,4-thiadiazole

¹H-NMR (300 MHz, CDCl₃)δ 7.43-7.42 (m, 1H), 7.35-7.30 (m, 4H), 7.13-7.10 (m, 2H), 5.43-5.39(m, 1H), 4.11-4.04 (m, 1H), 4.00-3.93 (m, 1H), 2.52-2.47 (m, 1H), 2.49 (s, 3H), 2.37-2.28 (m, 1H), 2.09-2.03 (m, 2H).

MH+ 491.

### Example 37

### 2-(5-(4-chlorophenyl)-1-(2,4-dichlorophenyl)-4-methyl-1H-pyrazol-3-yl)-5-methyl-1,3,4-thiadiazole

¹H-NMR (400 MHz, CDCl₃) δ 7.42 (d, J=1.84 Hz, 1H), 7.35-7.29 (m, 4H), 7.13-7.10 (m, 2H), 2.82 (s, 3H), 2.48 (s, 3H).

MH+ 435.

### Example 38

### 2-(5-(4-Chlorophenyl)-1-(2,4-dichlorophenyl)-4-methyl-1H-pyrazol-3-yl)-5-pentyl-1,3,4-thiadiazole

¹H-NMR (400 MHz, CDCl₃) δ 7.43-7.42 (m, 1H), 7.34-7.29 (m, 4H), 7.13-7.10 (m, 2H), 3.14 (t, J=7.8 Hz, 2H), 2.49 (s, 3H), 1.88-1.81 (m, 2H), 1.43-1.38 (m, 4H), 0.91 (t, J=7.3 Hz, 3H).

MH+ 491.

### Example 39

### 2-(5-(4-Chlorophenyl)-1-(2,4-dichlorophenyl)-4-methyl-1H-pyrazol-3-yl)-5-cycloheptyl-1,3,4-thiadiazole

¹H-NMR (400 MHz, CDCl₃) δ7.43-7.42 (m, 1H), 7.34-7.31 (m, 4H), 7.13-7.10 (m, 2H), 3.47-3.43 (m, 1H), 2.47 (s, 3H), 2.22-2.17 (m, 4H), 1.87-1.79 (m, 4H), 1.70-1.59 (m, 4H).

MH+ 517.

### Example 40

### 2-(5-(4-Chlorophenyl)-1-(2,4-dichlorophenyl)-4-methyl-1H-pyrazol-3-yl)-5-(2,4-dichlorophenyl)-1,3,4-thiadiazole

¹H-NMR (400 MHz, CDCl₃) δ 8.40 (d, J=8.4 Hz, 1H), 7.57 (d, J=2.2 Hz, 1H), 7.44 (d, J=1.8 Hz, 1H), 7.42 (d, J=2.2 Hz, 1H), 7.37-7.31 (m, 4H), 7.16-7.12 (m, 2H), 2.55 (s, 3H).

MH+ 564.

### Preparation of 1,2,4-oxadiazole

### Example 41

### 3-(5-(4-chlorophenyl)-1-(2,4-dichlorophenyl)-4-methyl-1H-pyrazol-3-yl)-5-cyclohexyl-1,2,4-oxadiazole

### Step 1: 5-(4-Chlorophenyl)-1-(2,4-dichlorophenyl)-4-methyl-1H-pyrazole-3-carboxamide

Thionyl chloride (1.3 ml, 22.0 mmol) was added to a solution of 5-(4-chlorophenyl)-1-(2,4-dichlorophenyl)-4-methyl-1H-pyrazole-3-carboxylic acid (2.09 g, 5.5 mmol) in toluene (50 ml) maintained at room temperature. The mixture was refluxed at 110□ for 2 hours and then cooled to room temperature. The resulting solution was evaporated and dried under a vacuum to produce crude 5-(4-chlorophenyl)-1-(2,4-dichlorophenyl)-4-methyl-1H-pyrazole-3-carbonyl chloride, which are not further purified. 27% aqueous ammonia (2 ml) was added to the solution of crude 5-(4-chlorophenyl)-1-(2,4-dichlorophenyl)-4-methyl-1H-pyrazole-3-carbonyl chloride in methylene chloride (10 ml) at 0□. After stirring 3 hours at room temperature, the resulting solution was quenched with saturated ammonium chloride which was extracted twice with 50 ml portions of ethyl acetate. After concentrating the extract by evaporation, the crude residue was purified using a silica gel column (hexane/ethyl acetate=1/1), to obtain the title compound (2.07 g, 99%) as a white solid.

### Step 2: 5-(4-Chlorophenyl)-1-(2,4-dichlorophenyl)-4-methyl-1H-pyrazole-3-carbonitrile

Added dropwise to a solution of 5-(4-chlorophenyl)-1-(2,4-dichlorophenyl)-4-methyl-1H-pyrazole-3-carboxamide (1.57 g, 4.1 mmol) obtained in Step 1 dissolved in dimethylformamide (10 ml) at 0□, was phosphoryl chloride (0.72 ml, 8.2 mmol). The resulting mixture was stirred for 20 minutes at 0□ and then stirred for 1 hour at room temperature. The reaction mixture was quenched with water at 0□, and an mixture was extracted with ethyl acetate (30 ml twice). After removing the solvent, 5-(4-chlorophenyl)-1-(2,4-dichlorophenyl)-4-methyl-1H-pyrazole-3-carbonitrile was obtained by column chromatography as a white solid (1.47 g, quantativly).

¹H NMR (400 MHz, CDCl₃) δ 7.40 (br s, 1H), 7.31-7.27 (m, 4H), 7.08-7.03 (m, 2H), 2.33 (s, 3H), 2.31 (t, J=7.8 Hz, 2H), 1.72 (m, 1H), 0.97 (t, J=7.3 Hz, 3H).

MH+ 463.

### Step 3: 5-(4-chlorophenyl)-1-(2,4-dichlorophenyl)-N-hydroxy-4-methyl-1H-pyrazole-3-carboximidamide

Potassium carbonate (0.58 g, 4.14 mmol) was added to a sealed tube containing a solution of 5-(4-chlorophenyl)-1-(2,4-dichlorophenyl)-4-methyl-1H-pyrazole-3-carbonitrile (1.0 g, 2.76 mmol) obtained in Step 2, hydroxylamine hydrochloride (0.39 g, 5.52 mmol) dissolved in MeOH (16 mL). The mixture was stirred vigorously and heated at 1001 for 16 hr. The white precipitate formed was filtered and washed twice with cold water. It was further dried under a high vacuum. The filtrate was extracted with chloroform (30 mL×2), dried over MgSO₄, filtered and evaporated under a vacuum, to obtain the title product in a compound amount of 1.04 g (2.63 mmol, 95%).

¹H NMR (400 MHz, DMSO-d₆) δ 9.77 (s, 1H), 8.27 (s, 1H), 7.69 (d, J=2.2 Hz, 1H), 7.63 (d, J=8.4 Hz, 1H), 7.51 (dd, J=2.2, 8.4 Hz, 1H), 7.41 (br d, J=8.6 Hz, 2H), 7.19 (br d, J=8.6 Hz, 2H), 5.49 (br s, 1H), 2.20 (s, 3H). MH+ 395.

### Step 4: 3-(5-(4-chlorophenyl)-1-(2,4-dichlorophenyl)-4-methyl-1H-pyrazol-3-yl)-5-cyclohexyl-1,2,4-oxadiazole

NMM (0.42 mL, 3.77 mmol) was added to a solution of 5-(4-chlorophenyl)-1-(2,4-dichlorophenyl)-N-hydroxy-4-methyl-1H-pyrazole-3-carboxamidamide (0.30 g, 0.75 mmol) obtained in Step 3, cyclohexylcarboxylic acid (97 mg, 0.75 mmol), HOBt (194 mg, 1.43 mmol) and EDCI (220 mg, 1.13 mmol) dissolved in DCM (7 mL). The mixture was stirred overnight at room temperature. After adding thereto EtOAc (30 mL), the mixture was successively washed with water, saturated NaHCO₃ solution (20 mL) and brine (20 mL). The organic layer was isolated, dried over MgSO₄, filtered, and concentrated under a vacuum, to obtain 5-(4-chlorophenyl)-N-(cyclohexanecarbonyloxy)-1-(2,4-dichlorophenyl)-4-methyl-1H-pyrazole-3-car boximidamide as a white solid, which was used in the next step without further purification. The crude material was dissolved in pyridine (4 mL), and was subjected to microwave irradiation at 180□ for 20 min., to remove volatile components under a vacuum. The residue was further purified by reverse phase preparative HPLC, to obtain the title compound as a white solid (67 mg, 0.137 mmol, 18% via two steps).

¹H NMR (300 MHz, CDCl₃) [symbol] 7.42-7.38 (m, 2H), 7.34-7.28 (m, 3H), 7.14-7.09 (m, 2H), 3.06 (m, 1H), 2.39 (s, 3H), 2.14 (m, 2H), 1.88-1.81 (m, 2H), 1.80-1.68 (m, 2H), 1.48-1.25 (m, 4H).

(M+Na)+ 509.

The following compounds of Examples 42 to 51 were obtained by repeating the procedure of Example 41.

### Example 42

### 3-(5-(4-chlorophenyl)-1-(2,4-dichlorophenyl)-4-methyl-1H-pyrazol-3-yl)-5-cyclopentyl-1,2,4-oxadiazole

¹H NMR (300 MHz, CDCl₃) [symbol] 7.42-7.38 (m, 2H), 7.34-7.28 (m, 3H), 7.14-7.09 (m, 2H), 3.45 (m, 1H), 2.39 (s, 3H), 2.20 (m, 2H), 2.06 (m, 2H), 1.86 (m, 2H), 1.72 (m, 2H).

(M+Na)+ 495.

### Example 43

### 3-(5-(4-chlorophenyl)-1-(2,4-dichlorophenyl)-4-methyl-1H-pyrazol-3-yl)-5-(pentan-3-yl)-1,2,4-oxadiazole

¹H NMR (300 MHz, CDCl₃) [symbol] 7.42-7.38 (m, 2H), 7.34-7.28 (m, 3H), 7.14-7.09 (m, 2H), 3.01 (m, 1H), 2.40 (s, 3H), 1.99-1.75 (m, 4H), 0.92 (t, J=7.5 Hz,3H).

(M+Na)+ 497.

### Example 44

### 5-benzyl-3-(5-(4-chlorophenyl)-1-(2,4-dichlorophenyl)-4-methyl-1H-pyrazol-3-yl)-1,2,4-oxadiazole

¹H NMR (300 MHz, CDCl₃) [symbol] 7.41-7.27 (m, 10H), 7.13-7.09 (m, 2H), 4.33 (s, 2H), 2.38 (s, 3H), 2.67-2.42 (m, 4H), 2.39 (s, 3H), 2.24-2.02 (m, 2H).

(M+Na)+ 517.

### Example 45

### 3-(5-(4-chlorophenyl)-1-(2,4-dichlorophenyl)-4-methyl-1H-pyrazol-3-yl)-5-cyclobutyl-1,2,4-oxadiazole

¹H NMR (300 MHz, CDCl₃) [symbol] 7.42-7.38 (m, 2H), 7.34-7.28 (m, 3H), 7.14-7.10 (m, 2H), 3.86 (m, 1H), 2.67-2.42 (m, 4H), 2.39 (s, 3H), 2.24-2.02 (m, 2H).

(M+Na)+ 481.

### Example 46

### 3-(5-(4-chlorophenyl)-1-(2,4-dichlorophenyl)-4-methyl-1H-pyrazol-3-yl)-5-cycloheptyl-1,2,4-oxadiazole

¹H NMR (300 MHz, CDCl₃) δ 7.42-7.37 (m, 2H), 7.32-7.27 (m, 3H), 7.13-7.10 (m, 2H), 3.25 (m, 1H), 2.39 (s, 3H), 2.24-2.15 (m, 2H), 2.01-1.81 (m, 4H), 1.65-1.60 (m, 6H)

(M+Na)+ 523.

### Example 47

### 3-(5-(4-chlorophenyl)-1-(2,4-dichlorophenyl)-4-methyl-1H-pyrazol-3-yl)-5-(cyclopropylmethyl)-1,2,4-oxadiazole

¹H NMR (400 MHz, CDCl₃) [symbol] 7.41-7.37 (m, 2H), 7.32-7.27 (m, 3H), 7.13-7.10 (m, 2H), 2.89 (d, J=6.88 Hz, 2H), 2.40 (s, 3H), 1.26 (m, 1H), 0.64 (m, 2H), 0.35 (AB q, J=5.04 Hz, 2H).

MH+ 459.

### Example 48

### 3-(5-(4-chlorophenyl)-1-(2,4-dichlorophenyl)-4-methyl-1H-pyrazol-3-yl)-5-(cyclopentylmethyl)-1,2,4-oxadiazole

¹H NMR (400 MHz, CDCl₃) [symbol] 7.41-7.37 (m, 2H), 7.32-7.27 (m, 3H), 7.13-7.10 (m, 2H), 2.98 (d, J=7.80 Hz, 2H), 2.46 (m, 1H), 2.40 (s, 3H), 1.98 (m, 1H), 1.91-1.82 (m, 2H), 1.72-1.52 (m, 4H), 1.36-1.25 (m, 2H).

MH+ 487.

### Example 49

### 3-(5-(4-chlorophenyl)-1-(2,4-dichlorophenyl)-4-methyl-1H-pyrazol-3-yl)-5-(cyclohexylmethyl)-1,2,4-oxadiazole

¹H NMR (400 MHz, CDCl₃) δ 7.41-7.37 (m, 2H), 7.32-7.27 (m, 3H), 7.13-7.10 (m, 2H), 2.86 (d, J=7.36 Hz, 2H), 2.40 (s, 3H), 1.98 (m, 1H), 1.80-1.62 (m, 5H), 1.33-1.10 (m, 5H).

MH+ 501.

### Example 50

### 3-(5-(4-chlorophenyl)-1-(2,4-dichlorophenyl)-4-methyl-1H-pyrazol-3-yl)-5-isopropyl-1,2,4-oxadiazole

MH+ 447.

### Example 51

### 3-(5-(4-chlorophenyl)-1-(2,4-dichlorophenyl)-4-methyl-1H-pyrazol-3-yl)-5-cyclopropyl-1,2,4-oxadiazole

MH+ 445.

### Preparation of Tetrazoles (Formula (Ie) and (If))

### Example 52

### 5-(5-(4-Chlorophenyl)-1-(2,4-dichlorophenyl)-4-methyl-1H-pyrazol-3-yl)-1-propyl-1H-tetrazole

### Step 1: 5-(5-(4-Chlorophenyl)-1-(2,4-dichlorophenyl)-4-methyl-1H-pyrazol-3-yl)-1H-tetrazole

A mixture of 5-(4-chlorophenyl)-1-(2,4-dichlorophenyl)-4-methyl-1H-pyrazole-3-carbonitrile (500 mg, 1.39 mmol), sodium azide (1.08 g, 16.6 mmol) and ammonium chloride (890 mg, 16.6 mmol) dissolved in dimethylformamide (3 ml) was placed in a microwave synthesizer tube, and subjected to microwave irradiation in a Biotage® Initiator was set at 180□. After 20 minutes, the mixture was quenched with water and extracted with ethyl acetate. The solvent of the extracted organic layer was evaporated off and the residue was purified by reverse phase preparative HPLC, to obtain 5-(5-(4-chlorophenyl)-1-(2,4-dichlorophenyl)-4-methyl-1H-pyrazol-3-yl)-1H-tetrazole (550 mg, 98%) as a white solid.

MH+ 404.

### Step 2: 5-(5-(4-Chlorophenyl)-1-(2,4-dichlorophenyl)-4-methyl-1H-pyrazol-3-yl)-1-propyl-1H-tetrazole

5-(5-(4-chlorophenyl)-1-(2,4-dichlorophenyl)-4-methyl-1H-pyrazol-3-yl)-1H-tetrazole (150 mg, 0.37 mmol) obtained in Step 1 was dissolved in dimethylformamide (1 ml), and potassium carbonate (250 mg, 1.85 mmol) and 1-iodopropane (0.07 ml, 1.1 mmol) were added thereto at room temperature. After stirring for 5 hours, the resulting solution was quenched with water (1 ml) and the mixture was three time extracted with 5 ml portion of ethyl acetate. The solvent of the extracted organic layer was evaporated under a reduced pressure and the residue was subjected to silica gel column chromatography (hexane/ethyl acetate=1/7 to hexane/ethyl acetate=1/5), to obtain two compounds, one of which was the title compound (61 mg, 37%) in the form of a white solid.

¹H NMR (400 MHz, CDCl₃) δ 7.51 (d, J=2.28 Hz, 1H), 7.36-7.32 (m, 2H), 7.29 (dd, J=8.72, 2.28 Mz, 1H), 7.18 (d, J=8.68 Hz, 1H), 7.14-7.10 (m, 2H), 4.78 (t, J=7.32 Hz, 2H), 2.46 (s, 3H), 2.03-1.93 (m, 20.94 (t, J=7.32 Hz, 3H).

MH+ 447.

### Example 53

### 5-(5-(4-Chlorophenyl)-1-(2,4-dichlorophenyl)-4-methyl-1H-pyrazol-3-yl)-2-propyl-2H-tetrazole

The title compound (62 mg, 37%) was the other compound obtained as a white solid in Example 52, which was relatively more polar than the compound of Example 52.

¹H NMR (400 MHz, CDCl₃) δ 7.41-7.38 (m, 2H), 7.33-7.28 (m, 3H), 7.15-7.12 (m, 2H), 4.66 (t, J=5.16, 2H), 2.46 (s, 3H), 2.17-2.07 (m, 2H), 1.01 (t, J=5.52 Hz, 3H).

MH+ 447.

The following compounds of Examples 54 to 79 were obtained by repeating the procedure of Example 52.

### Example 54

### 5-(5-(4-Chlorophenyl)-1-(2,4-dichlorophenyl)-4-methyl-1H-pyrazol-3-yl)-1-methyl-1H-tetrazole

¹H-NMR (400 MHz, CDCl₃) δ 7.49 (d, J=2.32 Hz, 1H), 7.35-7.22 (m, 4H), 7.14-7.10 (m, 2H), 4.39 (s, 3H), 2.47 (s, 3H).

MH+ 419.

### Example 55

### 5-(5-(4-Chlorophenyl)-1-(2,4-dichlorophenyl)-4-methyl-1H-pyrazol-3-yl)-2-methyl-2H-tetrazole

¹H NMR (400 MHz, CDCl₃) δ 7.40-7.38 (m, 2H), 7.33-7.27 (m, 3H), 7.14-7.12 (m, 2H), 4.44 (s, 3H), 2.46 (s, 3H).

MH+ 421.

### Example 56

### 5-(5-(4-Chlorophenyl)-1-(2,4-dichlorophenyl)-4-methyl-1H-pyrazol-3-yl)-1-ethyl-1H-tetrazole

¹H NMR (400 MHz, CDCl₃) δ 7.49 (d, J=1.71 Hz, 1H), 7.36-7.28 (m, 3H), 7.20 (d, J=6.51 Hz, 1H), 7.14-7.10 (m, 2H), 4.86 (q, J=5.52 Hz, 2H), 2.47 (s, 3H), 1.56 (t, J=5.52 Hz, 3H).

MH+ 433.

### Example 57

### 5-(5-(4-Chlorophenyl)-1-(2,4-dichlorophenyl)-4-methyl-1H-pyrazol-3-yl)-2-ethyl-2H-tetrazole

¹H NMR (400 MHz, CDCl₃) δ 7.41-7.38 (m, 2H), 7.33-7.27 (m, 3H), 7.15-7.12 (m, 2H), 4.75 (q, J=7.36 Hz, 2H), 2.46 (s, 3H), 1.71 (t, J=7.36 Hz, 3H).

MH+ 433.

### Example 58

### 1-Butyl-5-(5-(4-chlorophenyl)-1-(2,4-dichlorophenyl)-4-methyl-1H-pyrazol-3-yl)-1H-tetrazole

¹H NMR (400 MHz, CDCl₃) δ 7.49 (d, J=2.32 Hz, 1H), 7.35-7.27 (m, 3H), 7.19 (d, J=8.72 Hz, 1H), 7.14-7.10 (m, 2H), 4.82 (t, J=7.32 Hz, 2H), 2.46 (s, 3H), 1.97-1.89 (m, 2H), 1.55-1.33 (m, 2H), 0.91 (t, J=7.36 Hz, 3H). MH+ 460.

### Example 59

### 2-Butyl-5-(5-(4-chlorophenyl)-1-(2,4-dichlorophenyl)-4-methyl-1H-pyrazol-3-yl)-2H-tetrazole

¹H NMR (400 MHz, CDCl₃) δ 7.41-7.38 (m, 2H), 7.33-7.27 (m, 3H), 7.15-7.12 (m, 2H), 4.70 (t, J=7.32 Hz, 2H), 2.46 (s, 3H), 2.10-2.03 (m, 2H), 1.44-1.38 (m, 2H), 0.98 (t, J=7.32 Hz, 3H).

MH+459.

### Example 60

### 5-(5-(4-Chlorophenyl)-1-(2,4-dichlorophenyl)-4-methyl-1H-pyrazol-3-yl)-1-pentyl-1H-tetrazole

¹H NMR (300 MHz, CDCl₃) δ 7.50 (d, J=2.22 Hz, 1H), 7.36-7.27 (m, 3H), 7.19 (d, J=11.24 Hz, 1H), 7.14-7.10 (m, 2H), 4.81 (t, J=9.76 Hz, 2H), 2.46 (s, 3H), 1.97-1.83 (m, 2H), 1.32-1.25 (m, 4H), 0.86-81 (m, 3H).

MH+ 475.

### Example 61

### 5-(5-(4-Chlorophenyl)-1-(2,4-dichlorophenyl)-4-methyl-1H-pyrazol-3-yl)-2-pentyl-2H-tetrazole

¹H NMR (300 MHz, CDCl₃) δ 7.42-7.39 (m, 2H), 7.34-7.27 (m, 3H), 7.15-7.12 (m, 2H), 4.69 (t, J=7.14 Hz, 2H), 2.46 (s, 3H), 2.11-2.09 (m, 2H), 1.37-1.36 (m, is 4H), 0.92-0.88 (m, 3H).

MH+477.

### Example 62

### 5-(5-(4-Chlorophenyl)-1-(2,4-dichlorophenyl)-4-methyl-1H-pyrazol-3-yl)-1-isopropyl-1H-tetrazole

¹H NMR (300 MHz, CDCl₃) δ 7.49 (d, J=2.19 Hz, 1H), 7.36-7.27 (m, 3H), 7.20 (d, J=8.61 Hz, 1H), 7.14-7.10 (m, 2H), 5.68-5.61 (m, 1H), 2.44 (s, 3H), 1.66 (s, 3H), 1.63 (s, 3H).

MH+ 449.

### Example 63

### 5-(5-(4-Chlorophenyl)-1-(2,4-dichlorophenyl)-4-methyl-1H-pyrazol-3-yl)-2-isopropyl-2H-tetrazole

¹H NMR, (300 MHz, CDCl₃) δ 7.42-7.38 (m, 2H), 7.33-7.27 (m, 3H), 7.15-7.12 (m, 2H), 5.22-5.12 (m, 1H), 2.46 (s, 3H), 1.74 (s, 3H), 1.72 (s, 3H). MH+447.

### Example 64

### 5-(5-(4-Chlorophenyl)-1-(2,4-dichlorophenyl)-4-methyl-1H-pyrazol-3-yl)-1-cyclopentyl-1H-tetrazole

¹H NMR (400 MHz, CDCl₃) δ 7.49 (d, J=2.28 Hz, 1H), 7.35-7.27 (m, 3H), 7.20 (d, J=8.72 Hz, 1H), 7.14-7.09 (m, 2H), 5.75-5.67 (m, 1H), 2.44 (s, 3H), 2.24-2.16 (m, 4H), 2.04-1.98 (m, 2H), 1.78-1.68 (m, 2H).

MH+ 473.

### Example 65

### 5-(5-(4-Chlorophenyl)-1-(2,4-dichlorophenyl)-4-methyl-1H-pyrazol-3-yl)-2-cyclopentyl-2H-tetrazole

¹H NMR (400 MHz, CDCl₃) δ 7.41-7.36 (m, 2H), 7.33-7.27 (m, 3H), 7.15-7.11 (m, 2H), 5.34-5.27 (m, 1H), 2.44 (s, 3H), 2.37-2.24 (m, 4H), 2.04-1.94 (m, 2H), 1.84-1.73 (m, 2H).

MH+473.

### Example 66

### 5-(5-(4-Chlorophenyl)-1-(2,4-dichlorophenyl)-4-methyl-1H-pyrazol-3-yl)-1-cyclohexyl-1H-tetrazole

¹H NMR (400 MHz, CDCl₃) δ 7.52 (d, J=2.28 Hz, 1H), 7.36-7.26 (m, 3H), 7.16-7.10 (m, 3H), 5.25-5.17 (m, 1H), 2.42 (s, 3H), 2.19-1.30 (m, 10H).

MH+ 487.

### Example 67

### 5-(5-(4-Chlorophenyl)-1-(2,4-dichlorophenyl)-4-methyl-1H-pyrazol-3-yl)-2-cyclohexyl-2H-tetrazole

¹H NMR (400 MHz, CDCl₃) δ 7.41-7.38 (m, 2H), 7.35-7.27 (m, 3H), 7.15-7.10 (m, 2H), 4.84-4.76 (m, 1H), 2.45 (s, 3H), 2.31-1.31 (m, 10H).

MH+ 487.

### Example 68

### 1-Benzyl-5-(5-(4-chlorophenyl)-1-(2,4-dichlorophenyl)-4-methyl-1H-pyrazol-3-yl)-1H-tetrazole

¹H NMR (400 MHz, CDCl₃) δ 7.49 (d, J=2.28 Hz, 1H), 7.37-7.26 (m, 8H), 7.19 (d, J=8.24 Hz, 1H), 7.11-7.07 (m, 2H), 6.05 (s, 2H), 2.44 (s, 3H).

MH+ 496.

### Example 69

### 2-Benzyl-5-(5-(4-chlorophenyl)-1-(2,4-dichlorophenyl)-4-methyl-1H-pyrazol-3-yl)-2H-tetrazole

¹H NMR (400 MHz, CDCl₃) δ 7.45-7.43 (m, 2H), 7.39-7.27 (m, 8H), 7.13-7.10 (m, 2H), 5.85 (s, 2H), 2.43 (s, 3H).

MH+497.

### Example 70

### 2-((5-(5-(4-Chlorophenyl)-1-(2,4-dichlorophenyl)-4-methyl-1H-pyrazol-3-yl)-1H-tetrazol-1-yl)methyl)pyridine

¹H NMR (400 MHz, CDCl₃) δ 8.52 (d, J=4.53, 1H), 7.61-7.57 (m, 1H), 7.42 (d, J=2.32 Hz, 1H), 7.33-7.30 (m, 2H), 7.27-7.24 (m, 2H), 7.20-7.17 (m, 2H), 7.11-7.05 (m, 3H), 7.00 (d, J=7.80 Hz, 1H), 6.21 (s, 2H), 2.48 (s, 3H).

MH+ 496.

### Example 71

### 2-((5-(5-(4-Chlorophenyl)-1-(2,4-dichlorophenyl)-4-methyl-1H-pyrazol-3-yl)-2H-tetrazol-2-yl)methyl)pyridine

¹H NMR (400 MHz, CDCl₃) δ 8.61-8.59 (m, 1H), 7.71-7.66 (m, 1H), 7.40-7.37 (dd, J=5.04, 2.72 Hz, 2H), 7.33-7.26 (m, 4H), 7.18 (d, J=8.24 Hz, 1H), 7.14-7.11 (m, 2H), 6.03 (s, 2H), 2.45 (s, 3H).

MH+498.

### Example 72

### 3-((5-(5-(4-Chlorophenyl)-1-(2,4-dichlorophenyl)-4-methyl-1H-pyrazol-3-yl)-1H-tetrazol-1-yl)methyl)pyridine

¹H NMR (400 MHz, CDCl₃) δ 8.65 (d, J=1.84, 1H), 8.54 (dd, J=4.56, 1.36 Hz, 1H), 7.23-7.70 (m, 1H), 7.50 (d, J=2.32 Hz, 1H), 7.35-7.31 (m, 3H), 726-7.20 (m, 2H), 7.11-7.08 (m, 2H), 6.09 (s, 2H), 2.46 (2, 3H).

MH+ 496.

### Example 73

### 3-((5-(5-(4-Chlorophenyl)-1-(2,4-dichlorophenyl)-4-methyl-1H-pyrazol-3-yl)-2H-tetrazol-2-yl)methyl)pyridine

¹H NMR (400 MHz, CDCl₃) δ 8.76 (d, J=1.84 Hz, 1H), 8.62 (dd, J=5.04, 1.84 Hz, 1H), 7.79-7.76 (m, 1H), 7.39-7.36 (m, 2H), 7.33-7.27 (m, 4H), 7.13-7.10 (m, 2H), 5.88 (s, 2H), 2.43 (s, 3H).

MH+ 498.

### Example 74

### 4-((5-(5-(4-Chlorophenyl)-1-(2,4-dichlorophenyl)-4-methyl-1H-pyrazol-3-yl)-1H-tetrazol-1-yl)methyl)pyridine

¹H NMR (400 MHz, CDCl₃) δ 8.54-8.52 (m, 2H), 7.48 (d, J=2.28 Hz, 1H), 7.34-7.28 (m, 3H), 7.14-7.07 (m, 5H), 6.07 (s, 2H), 2.47 (s, 3H).

MH+ 496.

### Example 75

### 4-((5-(5-(4-Chlorophenyl)-1-(2,4-dichlorophenyl)-4-methyl-1H-pyrazol-3-yl)-2H-tetrazol-2-yl)methyl)pyridine

¹H NMR (400 MHz, CDCl₃) δ 8.64-8.62 (m, 2H), 7.40-7.13 (m, 7H), 7.14-7.11 (m, 2H), 5.87 (s, 2H), 2.45 (s, 3H). MH+496.

### Example 76

### 5-(5-(4-Chlorophenyl)-1-(2,4-dichlorophenyl)-4-methyl-1H-pyrazol-3-yl)-1-(cyclohexylmethyl)-1H-tetrazole

¹H NMR (300 MHz, CDCl₃) δ 7.51 (d, J=2.4 Hz, 1H), 7.36-7.26 (m, 3H), 7.18-7.09 (m, 3H), 4.68 (d, J=7.32 Hz, 2H), 2.46 (s, 3H), 2.04-2.00 (m, 1H), 1.68-0.90 (m, 10H).

MH+501.

### Example 77

### 5-(5-(4-Chlorophenyl)-1-(2,4-dichlorophenyl)-4-methyl-1H-pyrazol-3-yl)-2-(cyclohexylmethyl)-2H-tetrazole

¹H NMR (300 MHz, MeOH-d₄) δ 7.58-7.55 (m, 2H), 7.47-7.44 (m, 1H), 7.41-7.37 (m, 2H), 7.27-7.23 (m, 2H), 4.60 (d, J=7.14 Hz, 2H), 2.39 (s, 3H), 2.18-2.02 (m, 1H), 1.77-0.86 (m, 10H).

MH+501.

### Example 78

### 5-(5-(4-Chlorophenyl)-1-(2,4-dichlorophenyl)-4-methyl-1H-pyrazol-3-yl)-1-phenethyl-1H-tetrazole

¹H NMR (300 MHz, CDCl₃) δ 7.52 (d, J=2.37 Hz, 1H), 7.36-7.07 (m, 11H), 5.04 (t, J=7.68 Hz, 2H), 3.24 (t, J=7.86 Hz, 2H), 2.39 (s, 3H).

MH+508.

### Example 79

### 5-(5-(4-Chlorophenyl)-1-(2,4-dichlorophenyl)-4-methyl-1H-pyrazol-3-yl)-2-phenethyl-2H-tetrazole

¹H NMR (300 MHz, CDCl₃) [symbol] 7.42-7.39 (m, 2H), 7.34-7.26 (m, 6H), 7.25-7.19 (m, 2H), 7.16-7.11 (m, 2H), 4.92 (t, J=7.68 Hz, 2H), 3.41 (t, J=7.89 Hz, 2H), 2.45 (2, 3H).

MH+509.

### Example 80

### 5-(5-(4-Chlorophenyl)-1-(2,4-dichlorophenyl)-4-methyl-1H-pyrazol-3-yl)-1-(furan-2-ylmethyl)-1H-tetrazole

A mixture of 5-(5-(4-chlorophenyl)-1-(2,4-dichlorophenyl)-4-methyl-1H-pyrazol-3-yl)-1H-tetrazole (113 mg, 0.28 mmol) obtained in Step 1 of Example 52, furfuryl alcohol (0.05 ml, 0.56 mmol) and triphenylphosphine (146 mg, 0.56 mmol) was dissolved in tetrahydronfuran (3 ml) and cooled down to 0□. Then, diisopropyl azodicarboxylate (0.11 ml, 0.56 mmol) was slowly added thereto at 0□. After stirring for 2 hours at room temperature, the resulting solution was quenched with saturated ammonium chloride solution (1 ml). The mixture was extracted with ethyl acetate, and then the solvent of the organic layer was removed under a reduced pressure. The residue contained two regioisomers were separated by silica gel column chromatography (hexane/ethyl acetate=1/5), and repurified by reverse phase preparative HPLC, to obtain two compound, one of which was the title compound (22 mg, 16%).

¹H NMR (400 MHz, CDCl₃) δ 7.50-7.47 (m, 1H), 7.35-7.23 (m, 6H), 7.14-7.10 (m, 3H), 6.07 (s, 2H), 2.46 (s, 3H). MH+484.

### Example 81

### 5-(5-(4-Chlorophenyl)-1-(2,4-dichlorophenyl)-4-methyl-1H-pyrazol-3-yl)-2-(furan-2-ylmethyl)-2H-tetrazole

The title compound (24 mg, 18%) was the other regioisomer obtained as a white solid in Example 80, which is relatively more polar than the compound of Example 80.

¹H NMR (400 MHz, CDCl₃) δ 7.48-7.27 (m, 7H), 7.14-7.10 (m, 3H), 5.86 (s, 2H), 2.43 (s, 3H).

MH+485.

The following compounds of Examples 82 to 85 were obtained by repeating the procedure of Example 80.

### Example 82

### 5-(5-(4-Chlorophenyl)-1-(2,4-dichlorophenyl)-4-methyl-1H-pyrazol-3-yl)-1-(furan-3-ylmethyl)-1H-tetrazole

¹H NMR (400 MHz, CDCl₃) δ 7.50-7.51 (m 1H), 7.46 (s, 1H), 7.36-7.31 (m, 4H), 7.25-7.23 (m, 1H), 7.14-7.10 (m, 2H), 6.46 (s, 1H), 5.91 (s, 2H), 2.47 (s, 3H).

MH+484.

### Example 83

### 5-(5-(4-Chlorophenyl)-1-(2,4-dichlorophenyl)-4-methyl-1H-pyrazol-3-yl)-2-(furan-3-ylmethyl)-2H-tetrazole

¹H NMR (400 MHz, CDCl₃) δ 7.61 (s, 1H), 7.41-7.37 (m, 3H), 7.32-7.27 (m, 3H), 7.14-7.10 (m, 2H), 6.52 (m, 2H), 5.73 (s, 2H), 2.44 (s, 3H).

MH+484.

### Example 84

### 5-(5-(4-Chlorophenyl)-1-(2,4-dichlorophenyl)-4-methyl-1H-pyrazol-3-yl)-1-(thiophen-3-ylmethyl)-1H-tetrazole

¹H NMR (300 MHz, CDCl₃) δ 7.51 (d, J=2.19 Hz, 1H), 7.35-7.30 (m, 4H), 7.26-7.21 (m, 2H), 7.13-7.09 (m, 3H), 6.06 (s, 2H), 2.45 (s, 3H).

MH+501.

### Example 85

### 5-(5-(4-Chlorophenyl)-1-(2,4-dichlorophenyl)-4-methyl-1H-pyrazol-3-yl)-2-(thiophen-3-ylmethyl)-2H-tetrazole

¹H NMR (300 MHz, CDCl₃) δ 7.43-7.41 (m, 1H), 7.40-737 (m, 2H), 7.33-7.27 (m, 4H), 7.18 (dd, J=4.95, 1.29 Hz, 1H), 7.14-7.10 (m, 2H), 5.87 (s, 2H), 2.44 (s, 3H).

MH+501.

### Example 86

### 5-(5-(4-Chlorophenyl)-1-(2,4-dichlorophenyl)-4-methyl-1H-pyrazol-3-yl)-1-cyclopropyl-1H-tetrazole

Crude 5-(4-chlorophenyl)-1-(2,4-dichlorophenyl)-4-methyl-1H-pyrazole-3-carbonyl chloride (150 mg, 0.37 mmol) was dissolved in methylene chloride (3 ml) at 0□, and triethyl amine (0.05 ml, 1.11 mmol) and cyclopropyl amine (0.03 ml, 0.56 mmol) were slowly added thereto. The resulting solution was stirred at room temperature for 3 hours, quenched with saturated ammonium chloride solution, extracted with ethyl acetate and filtrated with magnesium sulfate. The crude solution was evaporated and dried under a reduced pressure, to obtain 5-(4-chlorophenyl)-N-cyclopropyl-1-(2,4-dichlorophenyl)-4-methyl-1H-pyrazole-3-carboxamide, which are not further purified. The resulting product was dissolved in benzene (3 ml), and phosphorus pentachloride (85 mg, 0.41 mmol) was added thereto at room temperature. The solution was stirred for 20 minutes, hydroazoic acid (1.0 ml, 2.0 M solution in benzene) was added thereto and maintained room temperature for overnight. For completion of the reaction, reaction solution was refluxed for 10 minutes and purified by reverse phase preparative HPLC, to obtain titled compound (83 mg, 50%) as a white solid.

¹H NMR (300 MHz, CDCl₃) δ 7.49-7.47 (m, 1H0, 7.34-7.22 (4H), 7.14-7.11 (m, 2H), 4.44 (m, 1H), 2.44 (s, 3H), 1.46-1.40 (m, 2H), 1.24-1.18 (m, 2H).

MH+445.

The following compounds of Examples 87 to 92 were obtained by repeating the procedure of Example 86.

### Example 87

### 5-(5-(4-Chlorophenyl)-1-(2,4-dichlorophenyl)-4-methyl-1H-pyrazol-3-yl)-1-cyclobutyl-1H-tetrazole

¹H NMR (400 MHz, CDCl₃) δ 7.50 (d, J=2.32 Hz, 1H), 7.35-7.29 (m, 3H), 7.22 (d, J=8.24 Hz, 1H), 7.13-7.09 (m, 2H), 5.75-5.67 (m, 1H), 2.86-2.75 (m, 2H), 2.62-2.53 (m, 2H), 2.43 (s, 3H), 2.03-1.85 (m, 2H).

MH+ 459.

### Example 88

### 5-(5-(4-Chlorophenyl)-1-(2,4-dichlorophenyl)-4-methyl-1H-pyrazol-3-yl)-1-cycloheptyl-1H-tetrazole

¹H NMR (300 MHz, CDCl₃) δ 7.42 (m, 1H), 7.36-7.26 (m, 3H), 7.07-7.04 (m, 2H), 6.91-6.88 (m, 1H), 4.13 (m, 1H), 2.37 (s, 3H), 2.08-2.02 (m, 2H), 1.75-1.52 (m, 8H), 1.27-1.22 (m, 3H), 0.93-0.82 (m, 3H).

MH+500.

### Example 89

### 1-tert-Butyl-5-(5-(4-chlorophenyl)-1-(2,4-dichlorophenyl)-4-methyl-1H-pyrazol-3-yl)-1H-tetrazole

¹H NMR (300 MHz, CDCl₃) δ 7.44-7.43 (m, 1H), 7.34-7.26 (m, 4H), 7.07-7.03 (m, 2H), 2.25 (s, 3H), 1.58 (s, 9H).

MH+460.

### Example 90

### 5-(5-(4-Chlorophenyl)-1-(2,4-dichlorophenyl)-4-methyl-1H-pyrazol-3-yl)-1-hexyl-1H-tetrazole

¹H NMR (300 MHz, CDCl₃) δ 7.43-7.42 (m, 1H), 7.32-7.23 (m, 2H), 7.07-7.04 (m, 2H), 6.94 (m, 1H), 3.41 (q, J=6.96 Hz, 2H), 2.38 (s, 3H), 1.63-1.25 (m, 7H), 0.91-0.85 (m, 4H).

MH+489.

### Example 91

### 5-(5-(4-Chlorophenyl)-1-(2,4-dichlorophenyl)-4-methyl-1H-pyrazol-3-yl)-1-octyl-1H-tetrazole

¹H NMR (300 MHz, CDCl₃) δ 7.43-7.41 (m, 1H), 7.31-7.24 (m, 3H), 7.10-7.03 (m, 2H), 6.94-6.92 (m, 1H), 3.41 (q, J=7.14 Hz, 2H), 2.38 (s, 3H), 1.62-0.85 (m, 15H).

MH+516.

### Example 92

### 1-Adamantyl-5-(5-(4-chlorophenyl)-1-(2,4-dichlorophenyl)-4-methyl-1H-pyrazol-3-yl)-1H-tetrazole

¹H NMR (300 MHz, CDCl₃) δ 7.47 (d, J=2.22 Hz, 1H), 7.34-7.28 (m, 4H), 7.15-7.12 (m, 2H), 2.42 (s, 6H), 2.20 (br, 3H), 2.12 (s, 3H), 1.76 (m, 6H).

MH+541.

### Preparation of Triazole (Formula (Id))

### Example 93

### 3-(5-(4-Chlorophenyl)-1-(2,4-dichlorophenyl)-4-methyl-1H-pyrazol-3-yl)-5-cyclopentyl-1H-1,2,4-triazole

A solution of 5-(4-chlorophenyl)-1-(2,4-dichlorophenyl)-4-methyl-1H-pyrazole-3-carbonitrile (300 mg, 0.83 mmol), cyclopentanecarbohydrazide (116 mg, 0.90 mmol) and potassium carbonate (57 mg, 0.41 mmol) dissolved in 1-butanol (2 ml) was placed in a in sealed tube, stirred room temperature for 10 minutes and then refluxed at 150□ for 1 days. The mixture was cooled down to room temperature and methanol (4 ml) was thereto, followed by filtering. The filtrate was purified by reverse phase preparative HPLC, to obtain title compound as a white solid.

¹H NMR (400 MHz, CDCl₃) δ 7.43 (d, J=2.32 Hz, 1H), 7.35-7.27 (m, 3H), 7.07-7.03 (m, 2H), 3.32-3.23 (m, 1H), 2.25 (s, 3H), 2.17-2.08 (m, 2H), 1.98-1.78 (m, 4H), 1.74-1.67 (m, 2H).

MH+ 472.

The following compounds of Examples 94 and 95 92 were obtained by repeating the procedure of Example 93.

### Example 94

### 3-(5-(4-Chlorophenyl)-1-(2,4-dichlorophenyl)-4-methyl-1H-pyrazol-3-yl)-5-cyclohexyl-1H-1,2,4-triazole

¹H NMR (400 MHz, CDCl₃) δ7.39-7.35 (m, 2H), 7.32-7.27 (m, 3H), 7.12-7.09 (m, 2H), 2.91-2.84 (m, 1H), 2.43 (s, 3H), 2.17-2.10 (m, 2H), 1.87-1.83 (m, 2H), 175-1.73 (m, 1H), 1.67-1.58 (m, 3H), 1.46-1.28 (m, 2H).

MH+ 486.

### Example 95

### 3-(5-(4-Chlorophenyl)-1-(2,4-dichlorophenyl)-4-methyl-1H-pyrazol-3-yl)-5-phenyl-1H-1,2,4-triazole

¹H NMR (400 MHz, CDCl₃) δ 8.21-8.18 (m, 2H), 7.47-7.42 (m, 4H), 7.35-7.29 (m, 4H), 7.14-7.12 (m, 2H), 2.54 (s, 3H).

MH+ 480.

### Pharmacological Test: In Vitro Activity Analysis

The compounds of the present invention were analyzed for their binding characteristics for CB₁ and CB₂ and the pharmacological activity thereof in accordance with the method disclosed in [Devane W A, Dysarz F A 3^{rd}, Johnson M R, Melvin L S and Howlett A C, Determination and characterization of a cannabinoid receptor in rat brain, Mol. Pharmacol., 34(5): 605-13 (1998)]. The analysis was performed using [³H]CP-55940 which is a selectively radioactivity-labeled 5-(1,1-dimethyheptyl)-2[5-hydroxy-2-(3-hydroxypropyl)-cyclohexyl]-phenol, purchased from PerkinElmer Life Sciences, Inc. (Boston, Mass., U.S.A.), through a rat CB-1 receptor binding protocol as follows.

The tissue obtained from the brain of SD rats was homogenized with a Dounce homogenate system in TME(50 mM Tris, 3 mM MgCl₂ and 1 mM EDTA, pH 7.4) at 4° C., and the homogenate was centrifuged at 48,000 g for 30 min. at 4° C. The pellet was resuspended in 5 ml of TME and the suspension was divided into aliquots and stored at −70° C. until its use in the following assay.

2 μl of the test compound was diluted in dimethylsulphoxide and was added to a deep well of a polypropylene plate, to which 50 μl of [³H]CP-55940 diluted in a ligand buffer solution (0.1% bovine serum albumin(BAS)+TME) was added. The tissue concentrations were determined by Bradford protein analysis, and 148 μl of brain tissue of the required concentration was added to the plate. The plate was covered and placed in a 30° C. incubator for 60 min, and then transformed on GF/B filtermat pretreated in polyethylenimine (PEI) using a cell harvester. Each filter was washed five times and dried at 60° C. for 1 hr. Then, the degree of radioactivity retained by the filter was measured using Wallac Microbeta™ (PerkinElmer Life Sciences, Inc., Massachusetts, U.S.A.) and the activity of the compound for inhibiting CB₁ receptor was determined therefrom.

While the invention has been described with respect to the specific embodiments, it should be recognized that various modifications and changes may be made by those skilled in the art to the invention which also fall within the scope of the invention as defined as the appended claims.

What is claimed is:

1. A compound of formula (I) or a pharmaceutically acceptable salt thereof: 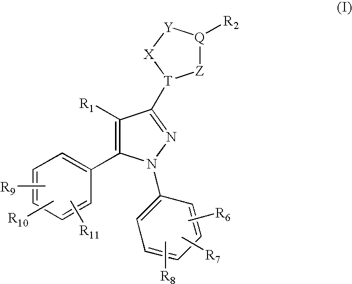
wherein:
R₁ is hydrogen, C₁₋₅ alkyl, substituted C₁₋₅ alkyl, C₂₋₄ alkenyl, substituted C₂₋₄ alkenyl, C₂₋₄ alkynyl, substituted C₂₋₄ alkynyl, or (CH₂)ₙ—C₃₋₅ carbocycle, n being 0 or 1;
R₂ is hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2;
R₃ and R₄ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or
R₃ and R₄, together with the nitrogen atom to which they are bonded, form a 4- to 10-membered saturated or unsaturated heterocyclic ring which is optionally substituted by one or more C₁₋₃ alkyl, benzyl, phenyl, C₁₋₃ alkoxy or halogen;
R₅ is phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutes;
R₆, R₇, R₈, R₉, R₁₀ and R₁₁ are each independently hydrogen, halogen, C₁₋₃ alkyl, C₁₋₃ alkoxy or trifluoromethyl;
X, Y and Z are each independently selected from the group consisting of —C(R₁₂)═, —O—, —N═, —N(R₁₃)— and —S— to form an aromatic heterocycle together with Q and T;
Q and T are each independently 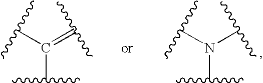
with the proviso that both Q and T can not be simultaneously 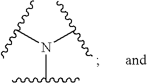
R₁₂ and R₁₃ are each independently hydrogen, carbocycle, substituted carbcycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, C₂₋₆ alkynyl optionally substituted by alkoxy or halogen, (CH₂)ₘ—C₃₋₆ carbocycle optionally substituted by alkoxy or halogen, or (CH₂)ₘ—R₅, m being 1 or 2, and R₅ having the same meaning as defined above.

2. The compound of claim 1, which is a compound of formula (Ia), (Ib), (Ic), (Id), (Ie) or (If): 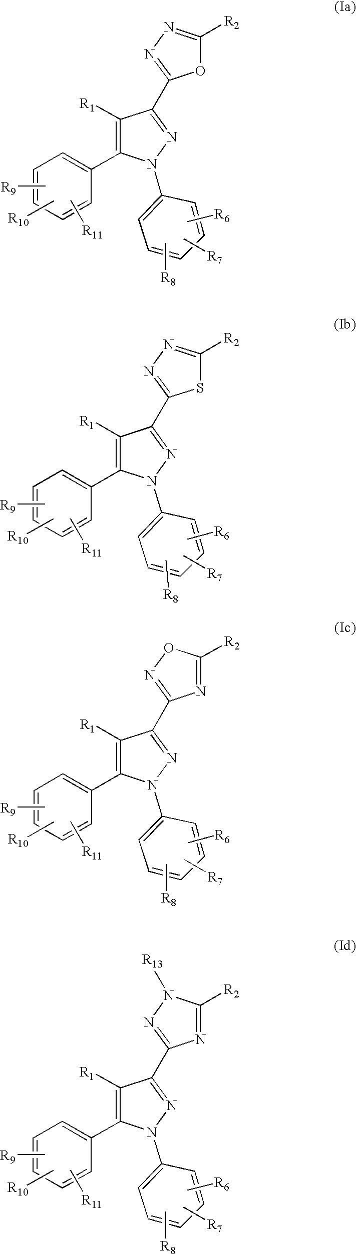 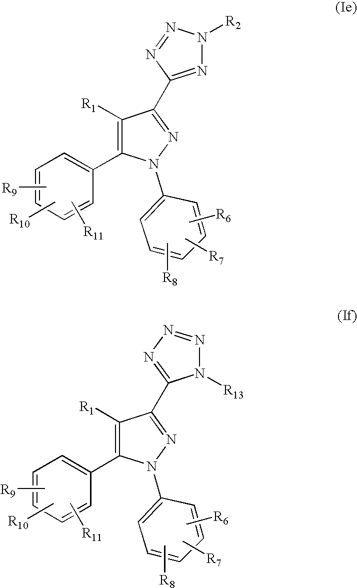
wherein, R₁, R₂, R₃, R₄, R₆, R₇, R₈, R₉, R₁₀, R₁₁ and R₁₃ have the same meanings as defined in claim 1.

3. A method for preparing the compound of formula (Ia) of claim 2, which comprises (i) reacting a carboxylic acid derivative of formula (5) with a hydrazide compound of formula (7) or a semicarbazide compound of formula (12) in the presence of a coupling agent in a solvent and (ii) cyclizing the resulting product using a dehydrating agent: 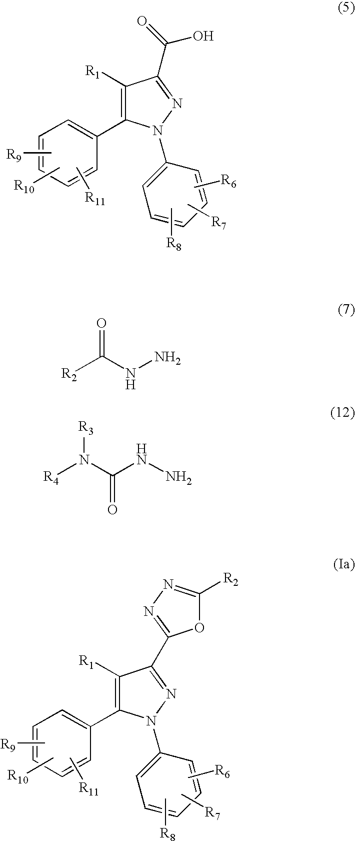
wherein, R₁, R₂, R₃, R₄, R₆, R₇, R₈, R₉, R₁₀ and R₁₁ have the same meanings as defined in claim 1.

4. The method of claim 3, wherein the cyclization is conducted using Burgess reagent as the dehydrating agent.

5. A method for preparing the compound of formula (Ib) of claim 2, which comprises (i) reacting a carboxylic acid of formula (5) with a hydrazide compound of formula (7) in the presence of coupling agents in a solvent and (ii) cyclizing the resulting product using a Lawesson's reagent: 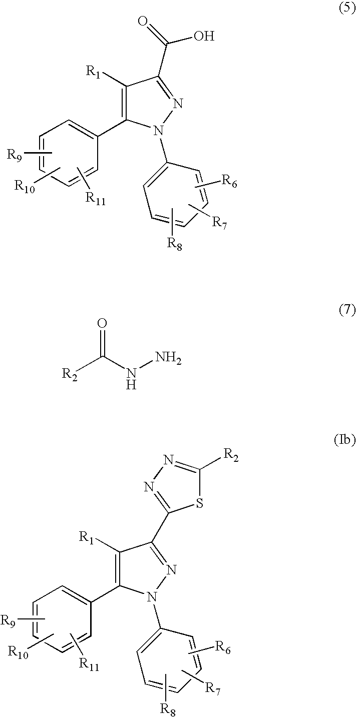
wherein, R₁, R₂, R₆, R₇, R₈, R₉, R₁₀ and R₁₁ have the same meanings as defined in claim 1.

6. A method for preparing the compound of formula (Ic) of claim 2, which comprises (i) reacting a nitrile intermediate of formula (19) with hydroxylamine in a solvent, (ii) acylating the resulting product with an activated carboxylic acid in the presence of a coupling agent, and (iii) cyclizing the acylated compound in the presence of a base: 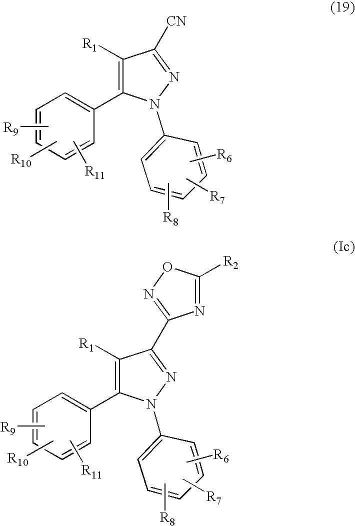
wherein, R₁, R₂, R₆, R₇, R₈, R₉, R₁₀ and R₁₁ have the same meanings as defined in claim 1.

7. A method for preparing the compound of formula (Id) of claim 2, which comprises reacting a nitrile intermediate of formula (19) with a hydrazide compound of formula (7) in the presence of a catalyst in a solvent: 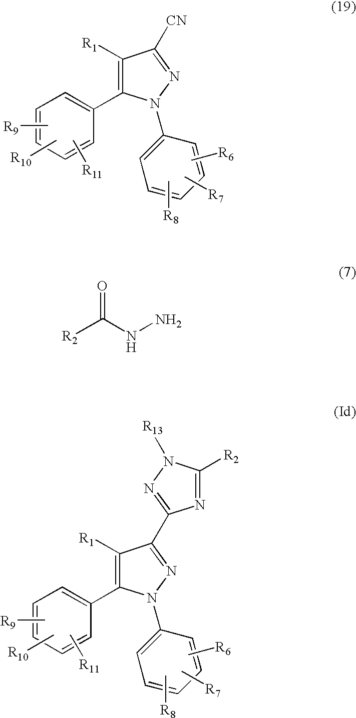
wherein, R₁, R₂, R₆, R₇, R₈, R₉, R₁₀, R₁₁ and R₁₃ have the same meanings as defined in claim 1.

8. A method for preparing the compound of formula (Ie) or (If) of claim 2, which comprises reacting a nitrile intermediate of formula (19) with sodium azide in the presence of a base in a solvent and optionally alkylating or acylating the resulting product: 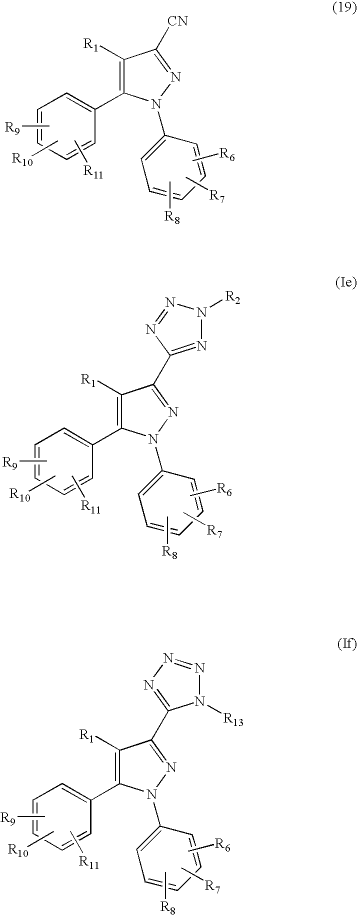
wherein, R₁, R₂, R₆, R₇, R₈, R₉, R₁₀, R₁₁ and R₁₃ have the same meanings as defined in claim 1.

9. A pharmaceutical composition comprising the compound of formula (I) of claim 1 as an active ingredient and a pharmaceutically acceptable carrier.

10. A method for preventing or treating obesity and obesity-related metabolic disorders in a mammal, which comprises administering the compound of formula (I) of claim 1 to the mammal.

11. A method for inhibiting cannabinoid CB₁ receptor in a mammal, which comprises administering the compound of formula (I) of claim 1 to the mammal.
